(19) United States
(12) Patent Application Publication
(10) Pub. No.: US 2015/0147774 A1
(43) Pub. Date: May 28, 2015

(54) EXPRESSION CONSTRUCT FOR YEAST AND A METHOD OF USING THE CONSTRUCT

(71) Applicant: National University of Singapore, Singapore (SG)

(72) Inventors: Jifeng YUAN, Singapore (SG); Chi Bun CHING, Singapore (SG)

(21) Appl. No.: 14/551,956

(22) Filed: Nov. 24, 2014

Related U.S. Application Data
(60) Provisional application No. 61/907,477, filed on Nov. 22, 2013.

(51) Int. Cl. C12N 15/90; C12N 15/81

(52) U.S. Cl. CPC C12N 15/905; C12N 15/81; C12N 2800/30
USPC 435/32; 435/320.1; 435/34

(57) ABSTRACT

The present disclosure provides an isolated linear expression cassette. The disclosed cassette comprises a bidirectional promoter; a first gene and a second gene that the genes are respectively and operably linked to one end of the bidirectional promoter; a terminator located immediately next to end of each gene; a 5′-homogolgous region to genomic context of interest and a 3′-homogolgous region to genomic context of interest respectively flanking 5′ end and 3′ end of the cassette; and a dominant selection marker residing within the construct and being arranged in between the flanking 5′-homogolgous region to genomic context of interest and a 3′-homogolgous region to genomic context of interest. Preferably, the cassette is capable of being integrated into genome of transformed yeasts upon being transported into the transformed cell.

This application claims priority under 35 U.S.C. §119 to U.S. application No. 61/907,477 filed on Nov. 22, 2013. The entire disclosure of the above-referenced application is hereby incorporated by reference.

### TECHNICAL FIELD

The present disclosure relates to an expression construct for integrating genes of interest into chromosomes of transformed yeast. Multiple copies of the disclosed expression construct can be incorporated to create duplication of the interested gene in a host cell and, subsequently, lead to rapid assembly of multiple-gene pathways for the overproduction of corresponding products encoded by the integrated gene in the transformed yeast. The pathway genes are assembled in a combinatorial manner with different gene copies and different chromosomal sites for modulating individual gene expressions to achieve a balanced metabolic flux for producing a compound-of-interest. The present disclosure also brings forth a process for implementing the disclosed construct and identifying the transformed yeast carrying preferred number of copy of the duplicated gene. The process can also be used for multiple-copy integration of single gene to create genetically stable strains capable of overproduction protein products with commercial values.

### BACKGROUND

Among microorganisms commonly exploited as microbial cell factories, Saccharomyces cerevisiae is particularly suitable to host diverse metabolite biosynthetic pathways. As an industrial microorganism commonly used in food and beverage production, S. cerevisiae is also a key model organism for fundamental molecular biology research, and it is the first eukaryotic organism with its genome completely sequenced (1). Based on the knowledge obtained throughout these years, various tools for pathway construction have been developed in S. cerevisiae, such as methods for controlling expression levels of heterologous genes, rapidly assembling of large biochemical pathways, and characterizing different chromosomal sites for heterologous gene expressions (2-5). Moreover, S. cerevisiae as a eukaryotic organism has the environment provided by an endomembrane such as the endoplasmic reticulum or mitochondrial inner membrane, for supporting the functional expression of membrane associated enzymes such as cytochrome P450s (6, 7). Another interesting feature of exploiting S. cerevisiae as a microbial cell factory is its capability for post-translational modifications of enzymes from plants and mammals (8).

Despite these advantages listed here, S. cerevisiae does not readily express multigene (polycistronic) transcriptional units to allow the coordinated expression of many genes within a compact operon. Thus it will be of great interest to develop new methods for rapid design and construction of large biochemical pathways in S. cerevisiae, especially for synthetic biology and metabolic engineering applications. Recently, Huimin Zhao and coworkers developed a new method, called ‘DNA assembler’ that enables rapid construction of large biochemical pathways in a one-step fashion in S. cerevisiae (4, 9). This method harnesses the high efficiency of recombination system of budding yeast, which has been widely used for plasmid construction, library creation and even for bacterial genome assembly (10-12). With overlapping homology more than 40 bp between individual cassettes, S. cerevisiae is capable of assembling a xylose utilization pathway and a zeaxanthin biosynthesis pathway into a vector, or integrated into the chromosome. Owing to the flaw stemmed from the design and arrangement of multiple genes along the expression vector used, DNA assembler, as described, may not be able to adjust gene copy numbers for modulating enzyme expression levels, where multiple copies of genes involved in the rate limiting steps are expected to further improve the pathway activity as demonstrated by some previous studies (6, 37). More importantly, different promoter and terminator flanking sequences for individual gene are required for functional expression of the gene of interest in the yeast transformed by the DNA assembler approach, in view of the fact that yeast does not readily express polycistronic genes. To assemble a multi-gene pathway, it is a must, in the DNA assembler approach, to avoid reuse of identical or similar regulatory elements. Reuse of identical regulatory elements in DNA assembler can result in occurrence of undesired internal recombination and end up with integration of incomplete biochemical pathways. Such restriction renders establishing of long biochemical pathways, which can sometimes involve ten or more individual genes, difficult considering that a new pair of promoter and terminator has to be included in the expression construct with employment for each additional gene. The use of different promoters for the expression of individual pathway genes will also hinder downstream process for refining promoter strengths with well-established promoter engineering techniques such as promoter libraries generated by error-prone PCR.

In a separate study, cocktail δ-integration coupled with different auxotrophic selection markers has been developed for constructing ratio-optimized cellulolytic enzyme expression in yeast (13). Different auxotrophic selection markers have to be used in constructing ratio-optimized cellulolytic enzyme expression in yeast for cocktail δ-integration. Application of auxotrophic selection markers, in the cocktail δ-integration, will typically provide single integration in which repeated rounds of genetic manipulation may be required in order to integrate more genes into the yeast chromosomes. Thus, cocktail δ-integration can be time-consuming yet has no guarantee of obtaining a transformed strain with the desired expression characteristics.

In view of the aforesaid, an alternative approach or expression system having improved features to at least tackle some of the shortcomings found in the abovementioned techniques is highly desired.

### SUMMARY

The present disclosure aims to offer a robust expression construct, which can be considered as a design principle as well, capable of carrying a gene of interest and being integrated into transformed yeasts. Particularly, the disclosed expression construct allows quick assembly of biochemical pathways into the transformed yeast, thus, enabling expression or overexpression of one or more compounds biosynthesized by the pathway of interest constructed therein. The disclosed expression construct also allows multiple-copy integration of a single gene to create genetically stable strains capable of overproduction of protein products with commercial values.

Another object of the present disclosure is to provide an expression construct incorporated with a dominant selection marker, which can be used to determine or estimate the degree of genome integration and facilitate identification of the desired transformed yeast expressing minimal amount of the markers positively corresponding to putative minimal copies of integrated genes. The dominant selection marker can be used to modulate the genome integration of a mixture of pathway cassettes for achieving one-step pathway assembly. Alternatively, individual pathway cassettes may also couple with different dominant selection markers to modulate integration copy number for individual cassettes.

The current disclosure also intends to present an expression construct permitting co-expression of multiple genes of interest, or multiple polypeptides encoded thereby, using a single or bidirectional promoter. The single, integrated or fused promoter system can be used for the production of protein products with high commercial values, and the resultant strains with chromosomal based expression will typically lead to more compact and stable overexpression of protein products over plasmid-based expression systems, as being disclosed in most of the conventional expression system. The bidirectional promoter architecture, to a certain extent, facilitates further promoter engineering for refining promoter strengths with further balanced metabolic flux. The expression construct can also be modified to couple with more genes by duplication of single promoter system or bidirectional promoter system. However, different pairs of promoter and terminator are used to prevent internal recombination to achieve higher degree of architecture.

A further object of the present disclosure is to offer a method of producing transformed yeasts. Preferably, the disclosed method favors integration of multiple copies of the aforesaid expression cassette in a single step fashion free from the need to perform repeated transformation steps as disclosed in some of the established approaches.

Still, the present disclosure aims to disclose a method utilizing selection pressure to act against one or more selection markers generated from the abovementioned expression cassette to identify the transformed yeast strain bearing the putative minimal number copies of integrated gene. In general, the selection pressure adopts a form which can be easily administrated towards the transformed yeast. More specifically, selection pressure may adversely influence growth of the transformed yeast such that only transformed yeast bearing minimal copies of integrated gene can survive in the presence of the pre-defined selection pressure.

At least one of the preceding objects is met, in whole or in part, by one or more embodiments in accordance with the present disclosure, which involves an isolated linear expression cassette. Several embodiments of the expression cassette comprise a bidirectional promoter; a first gene and a second gene that the genes are respectively and operably linked to one end of the bidirectional promoter; a terminator located immediately next to end of each gene; a 5′-Ty element and a 3′-Ty element respectively flanking 5′-end and 3′-end of the cassette; and a dominant selection marker residing within the construct and being arranged in between the flanking 5′-Ty element and the 3′-element, wherein the cassette is capable of being integrated into genome of a transformed yeast through the Ty-elements upon being transported into the transformed cell. Besides Ty elements, other genomic locations can be similarly implemented for the integration of expression cassettes to achieve multiple integration and pathway assembly. This can conveniently achieved by flanking different pairs of oligonucleotides, which is capable of targeting different genomic locations, during the process of PCR amplification of the genome integration cassettes.

In some other embodiments, the dominant selection marker of the disclosed expression cassette is an antibiotic selection marker such as ble, kanMX, natMX, hphMX or any modified selection marker derived thereof. Preferably, the dominant selection marker is coupled to and sandwiched in between a pair of site-specific recombination sequences.

In at least a few embodiments, the dominant selection marker is flanked with a pair of site-specific recombination sequences such as loxP sites to allow the recovery of selection marker defined in between. Alternatively, counter-selection marker such as I-SceI recognition sites can be embedded into the selection marker cassette to assist the recovery of selection marker.

According to a number of embodiments, the bidirectional promoter is a pair of promoters fused in opposite direction, and any functional promoter from native yeast cell, promoters of other origins and other organisms, or synthetic promoters can be similarly implemented to achieve the bidirectional promoter architecture. The bidirectional promoter allows the genes flanked at both ends or located, in a transcribable manner, downstream to the promoter to be transcribed by the transformed cells.

In a plurality of embodiments, the first gene and second gene are different from each other. More specifically, the first gene and second gene encode for different polypeptides. Preferably, genes encoding different pathway enzymes can be assembled with different combinations, which may have beneficial effect for balancing pathway flux for acquiring better yield of the compound of interest.

In accordance with several embodiments, the first gene and second gene can respectively encode ERG10, ERG13, tHMG1, ERG12, ERGS, ERG19, ID11 and ERG20 found in the mavalonate biosynthesis pathway. The disclosed cassettes can be applied to assemble or duplicate an orthogonal mevalonate pathway in the transformed yeasts. Additionally, the first gene and second gene can encode for enzyme responsible for isobutanol biosynthesis pathway, and the genes include ILV3, ILV5, ILV2, ARO10 and ADH7.

Another aspect of the present disclosure involves a method of transforming yeasts by way of genome integration. The method comprises the steps of transporting a mixture of linear expression cassettes into yeasts to produce transformed yeasts with at least one copy of the cassette integrated into genome of the yeasts; growing the yeast with a medium containing a reagent capable of reacting with a marker expressed from the linear expression cassette by the transformed yeasts to generate a signal thereof; and identifying the transformed yeasts based upon the generated signal. Preferably, the linear expression cassette includes a bidirectional promoter; a first gene and a second gene that the genes are respectively and operably linked to one end of the bidirectional promoter; a terminator located immediately next to end of each gene; a 5′-Ty element and a 3′-Ty element respectively flanking 5′ end and 3′ end of the cassette to enable integration of the cassette into genome of the transformed yeast upon transporting the cassette into the yeasts, and a dominant selection marker residing within the construct and being arranged in between the flanking 5′-Ty element and the 3′-element. Notably, the Ty elements can be modified or replaced by different sequences or genomic contexts to target the expression cassettes to other genomic locations.

In several embodiments, the marker is against an antibiotic being toxic to the yeasts and the reagent is the antibiotic; and, the identifying step further comprises selecting the transformed yeast with a minimal number of expression cassette integrated based upon resistance capacity of the transformed yeasts against the antibiotic of a predetermined concentration. The resistance capacity can be verified by growing the transformed yeast in the presence of the antibiotic of the predetermined concentration, the resistance capacity of the transformed yeast is substantially in positive correlation with the number of expression cassette integrated into the genome of the transformed yeasts. Other than ble selection marker confers phleomycin resistance, the cassettes of the described method may also use, kanMX, natMX and hphMX as the selection marker.

In at least a few embodiments, the selection marker is made removable with the use of a pair of site-specific recombination sequences. In such embodiments, the dominant selection marker is coupled to and sandwiched in between a pair of site-specific recombination sequences such as loxP sites to facilitate Cre recombinase-mediated marker recovery. More specifically, the employed selection marker can be removed or recovered after the cassettes are integrated into the yeast genome. Such flexibility offered in the present disclosure to recover or remove the marker permits successive rounds of transformation aiming to introduce more cassettes, preferably bearing the same or different genes from the first round of transformation, for tuning or improving the imparted traits of the transformed yeast. With the selection marker in the first batch of integrated cassettes removed, similar selection marker can be used again in the second batch of the constructed cassettes to be transported into the transformed yeast. Recoverable selection marker favors subsequent cassette or genome integration without exhausting the number of selection marker or facing limited options of applicable selection marker when the process involved is not merely two or three rounds of transformation but more. Recoverable selection marker found in the present disclosure facilitates genome integration events catalyzed by more than one round of transformation. Another benefit about marker recovery is that the strains with marker removed will have even more compact genome and gain greater efficiency in biosynthesis of the compound of interest. This can be attributed to the elimination of the expression of the selection marker, which consumes, or even competes for, carbon sources and all other building blocks as well as energy source available in the host. The expression cassette of the present disclosure offers better choice over other similar approaches developed based upon plasmid system, which is compelled to retain at least one selection marker in order to be functional.

For a plurality of embodiments, the first gene and second gene of the disclosed method are different and selected from the group consisting of ERG10, ERG13, tHMG1, ERG12, ERG8, ERG19, ID11 and ERG20.

According to some embodiments of the disclosed method, the first gene and the second gene of one linear expression cassette are different from another linear expression cassette in the mixture. More specifically, each of the linear expression cassettes contains two discrete genes and the two discrete genes carried in one cassette can be different from the two genes constructed on other cassettes; the rest of the elements in the constructed cassette are preferably the same except the genes carried. In certain circumstances, some genes encoding rate-limiting enzymes may be coupled with other genes at a much higher prevalence or frequency in order to achieve more integration events. The total number of genes found in the mixture of expression cassettes can be greater than two. For example, the number of genes can, but not limited to, range from 2 to 20.

A further aspect of the present disclosure includes a method of transforming yeast by way of genome integration. The method comprises the steps of transporting a plurality of linear expression cassettes into yeasts to produce transformed yeasts with at least one copy of the cassette integrated into the genome of the yeast that the linear expression cassette has a dominant selection marker against an antibiotic; and selecting the transformed yeast with a minimal number of expression cassette integrated based upon resistance capacity of the transformed yeasts against the antibiotic of a predetermined concentration. The resistance capacity can be verified, but not limited to, by growing the transformed yeast in the presence of the antibiotic of the predetermined concentration. The resistance capacity of the transformed yeast is substantially in positive correlation with the number of expression cassette integrated into the genome of the transformed yeasts.

According to some embodiments of the mentioned methods, the antibiotic against by the selection marker of ble gene is phleomycin and the predetermined concentration is at least 40 μg/mL for achieving and tracing genome integration events. Other antibiotic selection marker such as kanMX, natMX, or hphMX may be administrated at 100 μg/mL or more.

### BRIEF DESCRIPTION OF THE DRAWINGS

FIG. 1A is a schematic diagram of one embodiment of the disclosed cassette with 8-integration platform firstly built to a plasmid template comprising homologous sequences to δ-site of Ty elements and antibiotic selection marker to facilitate multiple integrations through modulating the selection pressure of antibiotics that the sequence from 5′-δ to 3′-δ will be amplified by PCR and used as genome integration cassette;

FIG. 1B is a schematic diagram illustrating one embodiment of the disclosed method for executing the novel δ-integration approach for assembling multiple-gene pathway into yeast chromosomes in a single step fashion, where high concentration of antibiotics is used to integrate the entire biochemical pathway into yeast chromosomes so that N for individual cassette is >1, MCS stands for multiple cloning sites and Ter stands for terminator;

FIG. 2 is a graph showing green fluorescence intensities for engineered strains obtained from library with cells adapted to various concentrations of antibiotics, where strains (1-4) were obtained from library treated with 40 μg/mL of phleomycin, strains (5-8) and strains (9-12) were from library supplemented with 80 and 160 μg/mL of phleomycin, respectively, and the green fluorescence intensities were recorded with excitation/emission at 476/512 nm. Experiments were performed in triplicate;

FIG. 3A is a graph showing isobutanol levels in engineered S. cerevisiae BY4742 strains under shake-tube condition, where alcohol levels were measured after 4 days cultivation using GC-MS and authentic alcohol compound was used for plotting standard curve for the quantification of isobutanol produced by engineered yeast cells and data represents the average and standard deviation of three independent experiments;

FIG. 3B is a chromatogram showing GC-MS result for the best producer M20 from library supplemented with high concentration of antibiotics (160 μg/mL of phleomycin);

FIG. 4 is a graph presenting qRT-PCR studies performed to determine overexpression of isobutanol pathway genes and the relative abundances of isobutanol pathway genes in each strain with respect to that of ACT1 (which encodes actin and serves as an internal control), the engineered strain M20 and the parental strain S. cerevisiae BY4742 were inoculated in SC media at initial OD600 of 0.05 and RNA was extracted from cell culture and further converted to cDNA after 12 h of cultivation;

FIG. 5A is a graph showing amorpha-4,11-diene levels in different engineered strains. All engineered strains were transformed with plasmid pYES2ADS harboring the codon-optimized version of amorpha-4,11-diene synthase from A. annua and cultured in SC medium with uracil dropped out and data represents the average and standard deviation of three independent experiments conducted with the amorpha-4,11-diene levels measured after 5 days;

FIG. 5B is a chromatogram showing GC-MS profile for compounds harvested from the best producer Y104;

FIG. 6 is a graph showing qRT-PCR analysis of the engineered strain Y104 to determine the expression levels of mevalonate pathway genes comparing to the parental strain that two cultures of each strain, the parental strain S. cerevisiae BY4742 and the engineered strain Y104, were inoculated in SC media at initial OD600 of 0.05 and harvested at exponential phase, data represents the relative abundances of mevalonate pathway genes in each strain with respect to that of ACT1 and experiment was carried out in triplicate;

FIG. 7 is a gel image verifying genome integration of the mevalonate pathway, where four-band pattern was observed with primer pair targeting at promoter pGAL10 and terminator tADH1 (From top to bottom: ERG13, ERG12, ERG19 and ID11) and four-band pattern was observed using primer pair targeting at promoter pGAL1 and terminator tCYC1 (From top to bottom: tHMG1, ERG8, ERG10 and ERG20);

FIG. 8 is a flowchart illustrating mevalonate pathway for carotenoid and amorpha-4,11-diene synthesis;

FIG. 9A is a graph presenting cell densities for engineered isobutanol producers; and

FIG. 9B is a graph showing peak area of alcohol levels during GC-MS analysis.

### DETAILED DESCRIPTION

The present invention may be embodied in other specific forms without departing from its arrangements, methods, or other essential characteristics as broadly described and claimed hereinafter. The described embodiments related to an expression construct and a transformation method are to be considered in all respects only as illustrative, and not restrictive. The scope of the invention is, therefore, indicated by the appended claims, rather than by the disclosed description. All changes that come within the meaning and range of equivalency of the claims are to be embraced within their scope.

Unless specified otherwise, the terms “comprising” and “comprise” as used herein, and grammatical variants thereof, are intended to represent “open” or “inclusive” language such that they include recited elements but also permit inclusion of additional, un-recited elements.

As used herein, the phrase “in embodiments” means in some embodiments but not necessarily in all embodiments.

As used herein, the terms “approximately” or “about”, in the context of concentrations of components, conditions, other measurement values, etc., means +/−5% of the stated value, or +/−4% of the stated value, or +/−3% of the stated value, or +/−2% of the stated value, or +/−1% of the stated value, or +/−0.5% of the stated value, or +/−0% of the stated value.

As used hereinafter, the terms “promoter”, “gene”, “Ty-element”, “terminator”, “dominant selection marker” shall refer to nucleotides sequences arranged in tandem along the disclosed expression construct, as an isolated or an integrated form, to carry out the function known, but not limited to, by one skilled artisan in the field as the term denoted. For instance, “Ty-element” refers to nucleotide sequences of non-viral retroelements or retrotransposons typically found in the nucleus of budding yeast (Saccharomyces cerevisiae) that “Ty elements” occur in several related forms and are designated Ty1, Ty2, Ty3, and Ty4, etc. The elements are flanked by long terminal direct repeats (251-371 bp) that are designated as δ for Ty1 and Ty2, σ for Ty3 and τ for Ty4. The design of homologous region to Ty elements has to be >40 bp for effective cassette integration into yeast chromosomes, albeit longer homologous region was found to be more effective for rapid pathway assembly.

The term “promoter” used herein throughout the specification shall refer to a site or a region in the cassette having a nucleotide sequence for binding of the transcriptase enzyme (RNA polymerase), transcription factor complexes, or regulatory elements of the transformed cell to facilitate expression of the gene operably located downstream of the promoter. The “bidirectional promoter” may further refer to a promoter region interposing between a bidirectional gene pair in which two genes, adjacent to the bidirectional promoter, coded on opposite strands, with their 5′ ends oriented toward one another. The two genes, each flanking at respective end of the bidirectional promoter, are preferably co-expressed in the same cassette.

The term “gene” as used herein may refer to a DNA sequence with functional significance. It can be a native nucleic acid sequence, or a recombinant nucleic acid sequences derived from natural source or synthetic construct. The term “gene” may also be used to refer to, for example and without limitation, a cDNA and/or an mRNA encoded by or derived from, directly or indirectly, genomic DNA sequence.

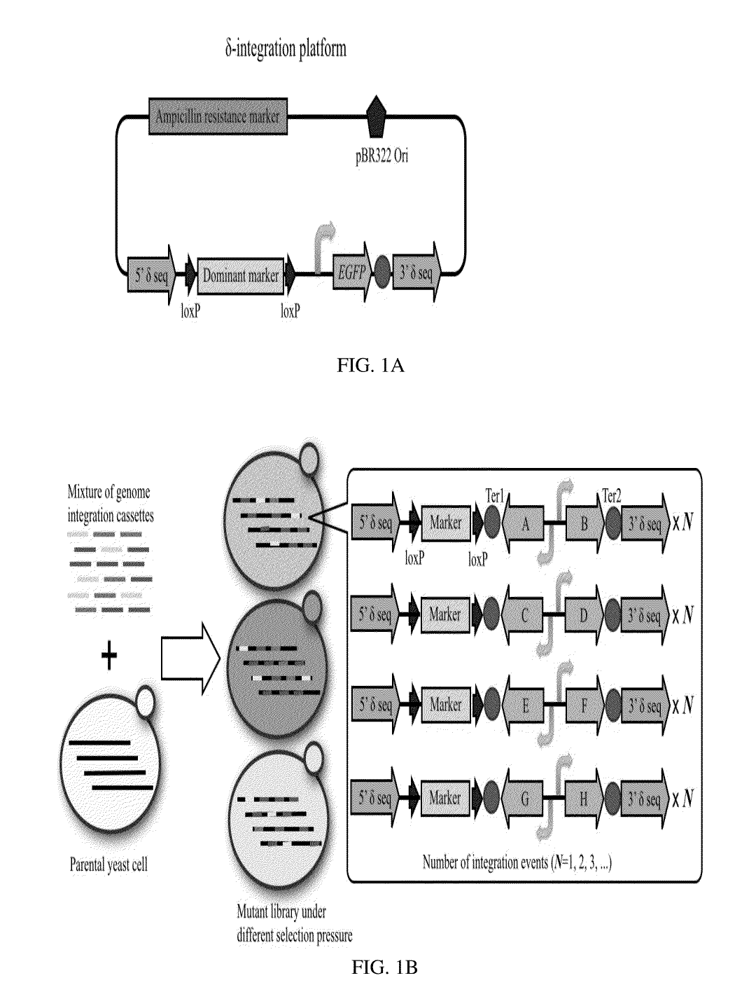

A plurality of embodiments of the present disclosure, as illustrated in FIG. 1A, involve an isolated linear expression cassette being configured to carry at least a pair of genes of interest and integrate the borne genes into genome of a host cell. Preferably, the host cell subjected for the genome integration is S. cerevisiae or other yeast strains featuring high recombination efficiency. To facilitate genome integration, the 5′-end and the 3′-end of the cassette are flanked with homologous region from long terminal repeats of retrotransposon elements or other genomic contexts. The long terminal repeats of retrotransposons elements are highly repetitive elements with known polynucleotide sequences from either active retrotransposons or remnants of long terminal repeats. The highly repetitive long terminal sequences from retrotransposon elements are readily to be used for integration of cassettes into yeast chromosomes, whereas other genomic contexts such as non-essential genes or other neutral sites can also be similarly implemented for the integration of cassettes. The present disclosed cassette defines the genes of interest and other transcription-regulatory elements in between the flanked homologous region, such as a 5′-retrotransposon element and a 3′-retrotransposon element, that fusion of the retrotransposon elements into the genome of the transformed yeast inevitably integrates the genes of interest defined therebetween into the genome. In more specific, the cassette is capable of being integrated into genome of the transformed yeasts through the Ty-elements upon being transported into the transformed cell. With the aid of the highly repetitive retrotransposon elements, transporting multiple copies of the disclosed cassette into the transformed yeasts shall lead to, theoretically, multiple genome integration of the transported copies. The retrotransposon elements applicable in the present disclosure for construction of the cassettes can be Ty1/1, Ty3, or Ty4 of Ty-elements. More preferably, δ sequence or δ types Ty-element are used through other types of Ty-elements or genomic contexts can be similarly implemented. As all the constructed cassettes are intercepted with essential genes, the cassettes are less likely to be popped-out, discharged or rejected by the host.

Referring further to FIG. 1B, several embodiments of the disclosed cassette carries a pair of genes for being transported and integrated into the genome of the yeasts. Present disclosure interposes a bidirectional promoter between the two genes, a first gene and a second gene, which are respectively and operably linked to one end of the bidirectional promoter. The first gene can be the element A, C, E or G, while the second gene is B, D, F, or H as shown in FIG. 1B. The two genes are co-regulated and co-expressed through the operably linked bidirectional promoter. More specifically, the bidirectional promoter realizes transcription of the two adjacently located genes in opposite direction once the constructed cassette is integrated into the genome. Each of the genes in fact is located, in a transcribable manner, downstream to the bidirectional promoter despite FIG. 1B showing the genes are respectively positioned upstream and downstream of the bidirectional promoter for brevity reason. Utilization of the bidirectional promoter architecture substantially reduces the number of promoter libraries required for potential future refinement of promoter strengths to control expression of each gene integrated thereof. The pairwise arrangement of the genes facilitates modular optimization of pathway gene expression. The disclosed construct facilitates one-step pathway assembly and enables integration of the transported cassette to take place in different area of the genome without substantial interference towards one another. The like feature, in turn, frees the disclosed cassette from the need of deploying multiple distinct promoters/terminators for expressing different integrated genes. More preferably, the bidirectional promoter can be formed by joining or fusing two of individual promoters in an opposing direction or fashion. The individual promoter can be used to generate the bidirectional promoter is any functional promoter from native yeast cells, promoter of other origins and organisms, or synthetic promoters.

As shown in the examples provided below, the disclosed cassette is configured to allow transportation and integration of multiple genes through a plurality of cassette types. Each cassette type may bear different gene pairs to assemble a partial or complete biochemical pathway within the transformed yeasts. In a number of embodiments, the gene pair, the first and second gene, incorporated to the disclosed cassettes can be ID11, tHMG1, ERG 8, ERG10, ERG12, ERG13, ERG19 and ERG 20 to at least partially build one or more additional mevalonate pathway inside the transformed yeast. More specifically, the genome integration of the heterologous genes are deemed equivalent to duplication of the genes of interest in the host that it generally results in overexpression of the enzymes and the like polypeptides, further followed by overproduction of the compounds catalyzed by the overexpressed enzymes. Likewise, in some other embodiments, the disclosed cassette carries a pair of genes applicable for isobutanol production in yeast. The genes associated to isobutanol synthesis and can be constructed to the cassette are ARO10, ADH7, ILV2, ILV3 and ILV5. Moreover, the disclosed cassette is configurable to assemble all other pathways or integrate other genes into the transformed yeasts to biosynthesize various compounds of interest. The first gene and second gene in the same cassette are preferably different from each other though the two genes can be the same in a few embodiments. To enable effective translation process, genes of interest will have prefixed Kozak sequence introduced into the primer region during the PCR amplification process.

In addition to the bidirectional promoter, the disclosed cassette preferably includes a terminator located immediately next to end of each gene. The terminator or terminator sequence signifies end of transcription and release of the transcribed mRNA from the template for peptide translation. According to the preferred embodiments, ADH1 and CYC1 terminators are used. In addition to that, all other terminator sequences can be similarly implemented in further embodiments of the disclosed construct. The employment of different terminators, other than ADH1 and CYC1, will be favorable for successive rounds of transformation and genome integration to introduce other genes of interest and avoid any unnecessary homologous recombination between the previous integration events.

According to a number of preferred embodiments, the disclosed cassette has a dominant selection marker resided within the construct that the selection marker is located in between the flanking 5′-Ty element and the 3′-Ty element in a fashion without interrupting transcription of the gene pair incorporated into the cassette. FIG. 1B exemplifies one embodiment of the disclosed cassette with the selection marker disposed downstream, in relation to the manner which transcription occurs, of the first gene. Other embodiments of the disclosed cassette may have the selection marker located after the second gene, in between the bidirectional promoter and the first gene or in between the bidirectional promoter and the second gene. The dominant selection marker described herein may refer to an antibiotic selection marker like ble, kanMX, natMX or hphMX. In the more preferred embodiments, the cassette contains antibiotic selection marker, which encoded for an enzyme to react against one or more antibiotic toxic to the host. Presence of the antibiotic selection marker in the host by way of genome integration permits continuous growth of the transformed yeast in a culture medium dosed with the antibiotic. On the other hand, unsuccessful genome integration or less integration events ensure extermination or poor growth of the cultured yeasts in the medium containing the antibiotic. Incorporation of the antibiotic selection marker allows the transformed yeast integrated with the disclosed cassette to be visually identified by spotting colonies formed on the antibiotic administered medium. Apart from that, the dominant selection marker of the disclosed cassette is coupled to and sandwiched in between a pair of site-specific recombination sequences in several preferred embodiments. The dominant selection marker interposing between the paired site-specific recombination sequences is made to be removable from the integrated cassettes. These site-specific recombination sequences are special polynucleotide sequences recognizable by corresponding enzyme or regulatory element to act on. The site-specific recombination sequences can be loxP sites recognizable and capable of reacting with Cre recombinase or other equivalents to carry out recombinase-mediated marker recovery. Preferably, other counter-selection marker may also be included between the paired site-specific recombination sequences to facilitate the marker recovery. Meganuclease recognition sites of I-SceI from the mitochondria of baker's yeast, I-CreI from the chloroplasts of Chlamydomonas reinhardtii and I-DmoI from the archaebacterium Desulfurcoccus mobilis will be of interest to serve as counter-selection marker. Alternatively, CRISPR/Cas9-mediated counter-selection using guide RNA targeting at the selection marker can also be of interest to materialize complete marker recovery.

Further, the disclosed cassette may also be combined with an element of signal peptide to the encoded polypeptides or proteins to be transported to mitochondria or other compartments of the transformed yeasts and thereby utilizing resources or precursors found to biosynthesize the compound of interest. Preferably, the mentioned element is a N-terminal or C-terminal localization signal or targeting signaling sequence fused to the polypeptide constructed to the cassettes. The polypeptide, or preferably enzyme, transcribed and translated from the integrated gene will be tagged with a short peptide sequence, encoded by the targeting sequence fused, prompting the host to move the newly created enzyme to the targeted locations, preferably mitochondria of the host. The mitochondrial targeting sequence applicable in the disclosed cassette can be from COX4, COQ3, etc. Besides of the mitochondrial targeting sequence, other sequences targeting different locations in the transformed yeasts can be used too. For example, the like targeting sequence may direct the synthesized polypeptides to be transported to nucleus, and transported to or retained at endoplasmic reticulum. One skilled artisan should appreciate the fact that full sequence may not be required in certain circumstances to enable the transportation, targeting sequences may be partially truncated to ensure that the constructed cassettes remain within the preferred length or size.

In accordance with a few embodiments, the cassette may be designed to carry at least an additional gene, a third gene, arranged further downstream to the first and/or second genes. This third gene operably couples to a secondary promoter and a secondary terminator, set aside the bidirectional promoter, to form an extra transcribable segment on the disclosed cassette. The additional gene or third gene resided in the segment is made fully transcribable in view of the secondary promoter and terminator fused thereto. The disclosed cassette preferably places the additional transcribable segment next to the terminator of the first and/or second gene. The segment should begin with the secondary promoter then the third gene and finally end with the secondary terminator. Such embodiments allows the integration and expression three or more genes in a single copy of the constructed cassette. The higher architecture design allows modular optimization for the pathway of interest.

It is preferred to build and store the template of the disclosed cassette in one or more plasmids so that multiple copies of the disclosed cassette can be generated by way of polymerase chain reaction (PCR). Besides multiple steps of restriction and ligation to insert the preferred elements into the plasmid, numerous approaches such as overlapping PCR, circular polymerase extension cloning (CPEC), Gibson DNA assembly or sequence and ligase independent cloning (SILC) can be similarly implemented as well for the assembly of the expression construct. The examples disclosed in the below description merely present one of the possible ways to construct or build the intermediate plasmid bearing the template of the disclosed cassette and any modifications made thereof shall not depart from the scope of the present disclosure.

Pursuant to another aspect of the present disclosure, a method of transforming yeasts by way of genome integration employing the aforementioned cassette is disclosed herein. The disclosed method essentially comprises transporting a plurality of linear expression cassettes into yeasts to produce transformed yeasts with at least one copy of the cassette integrated into genome of the yeasts, growing the yeast with a medium containing a reagent capable of reacting with a marker expressed from the dominant selection marker by the transformed yeasts to generate a signal thereof; and identifying the transformed yeasts based upon the generated signal. Preferably, the linear expression cassette comprises a bidirectional promoter; a first gene and a second gene that the genes are respectively and operably linked to one end of the bidirectional promoter; a terminator located immediately next to end of each gene; a 5′-Ty element and a 3′-Ty element respectively flanking 5′ end and 3′ end of the cassette to enable integration of the cassette into genome of the transformed yeast upon transporting the cassette into the yeasts; and a dominant selection marker residing within the construct and being arranged in between the flanking 5′-Ty element and the 3′-element.

As setting forth in the foregoing, the plurality of linear expression cassette to be transported can be acquired and amplified from a number of templates or intermediate plasmids storing the templates. It is important to note that the plurality of linear expression cassettes, in most embodiments, is a mixture of different cassettes types as shown in FIG. 1B, each type of the cassettes carries a pair of genes being distinctive from other cassette types. Specifically, the first gene and the second gene of one linear expression cassette or cassette type are different from another linear expression cassette or cassette type. Each cassette type can be amplified from the corresponding template plasmid under one or more PCR reactions. For some embodiments, each cassette types are prepared in equimolar before bringing into contact with the preferred yeast strain for transformation. Yet, it is feasible too in the disclosed method to mixed each cassette type in various molar or amount to substantially tune the integration of the gene copies and the polypeptides or enzyme expressed thereof. For example, the disclosed method may prepare a commonly known low expression polypeptide with greater amount in the mixture, in relation to other cassette types, to boost higher genome integration of this particular gene or genes, greater genome integration of this particular gene shall result increased expression of the encoded polypeptides by the host.

Despite examples provided hereinafter show that the transporting step or the yeast transformation can be achieved by way of electroporation, there are other procedures or processes applicable to move the cassettes across the cellular membrane into the host. Other practices such as lithium acetate/poly(ethyleneglycol) method, biolistics, lipofection, calcium phosphate transfection, diethylaminoethyl cellulose (DEAE-C) transfection and microinjection can be employed, with or without known modification, for the cassettes transportation.

Similarly, retrotransposons elements or transposable elements with known polynucleotide sequences are employed in the disclosed method to flank both 5′-end and 3′-end of the cassettes. The retrotransposon elements confer the cassettes with the capability to integrate and/or merge into the genome of the host cell. In more specific, the retrotransposon elements applicable in the disclosed method for producing the transformed yeast can be Ty1/2, Ty3, or Ty4. More preferably, δ sequence or δ types of Ty-element are used to yield better integration efficacy as explained in the foregoing description. Besides these highly repetitive retrotransposon elements, other genomic contexts can also be implemented for the integration of cassettes, by simply replacing homologous sequences of Ty elements to other genomic sequences.

Preferably, the two genes in the cassette to be integrated into the yeast genome are co-regulated and co-expressed through the operably linked bidirectional promoter. The linear expression cassettes have each of the incorporated genes arranged, in a transcribable manner, downstream to the bidirectional promoter. Adopting the use of a bidirectional promoter substantially reduces the number of promoter libraries required for future refinement of promoter strengths to tune the expression level of each gene integrated thereof. Preferably, the bidirectional promoter library with different transcriptional strengths can be generated using error-prone PCR. According to the preferred embodiments, the bidirectional promoter can be formed by joining or fusing two of individual promoters in an opposing direction or fashion. The individual promoter can be used to generate the bidirectional promoter is any functional promoter from native yeast cells, promoter of other origins and organisms, or synthetic promoters.

In a number of preferred embodiments of the disclosed method, the marker encodes a protein capable of imparting resistance, to the transformed yeasts, against an antibiotic being toxic to the transformed yeasts and the reagent is the antibiotic. Inventors of the present disclosure found that the yield of this antibiotic-resistance protein in the transformed yeast is positively correlated to both resistance capacity of the transformed yeast towards the antibiotic and the degree of genome integration, more precisely the number of successfully integrated cassettes. The inventors further exploit such findings of substantial correlation to select transformed yeast with the estimated minimal genome integration rate or minimal copies of integrated cassette by means of selection pressure applied to the transformed yeast in a culture medium. In more specific, it is believed that only transformed yeast carrying a minimal number of integrated cassette, which theoretically corresponds to the resistance capacity and the amount of produced resistance protein, can survive and grow in a medium containing a predetermined dose of the antibiotic. By adjusting or regulating the antibiotic concentration in the medium, the practitioner of the disclosed method can conveniently identify transformed strain with relatively greater genome integration, which should substantially equal to better compound yielder or producer. Thus, the identifying step may further include selecting the transformed yeast with a minimal number of expression cassette integrated based upon resistance capacity of the transformed yeasts against the antibiotic of a predetermined concentration by growing the transformed yeast in the presence of the antibiotic of the predetermined concentration, that the resistance capacity of the transformed yeast is substantially in positive correlation with the number of expression cassette integrated into the genome of the transformed yeasts. In addition, the dominant selection marker of the cassette is coupled to and sandwiched in between a pair of site-specific recombination sequences in several preferred embodiments of the disclosed method. The dominant selection marker interposing between the paired site-specific recombination sequences are made to be removable from the integrated cassettes. The pair of site-specific recombination sequences can be loxP sites recognizable and capable of reacting with Cre recombinase or other equivalents to carry out recombinase-mediated marker recovery. It is important to note that the disclosed method utilizes modified 8-integration with antibiotic selection marker for easy modulation or manipulation of the integration events to achieve ratio-optimized expression of pathway genes in one-step fashion, in opposition to cocktail δ-integration deploying different auxotrophic selection markers just to construct ratio-optimized cellulolytic enzyme expression in yeast (13). Auxotrophic selection marker will typically result in single integration and generally take several rounds of genetic manipulation to introduce more genes to be integrated into the yeast chromosomes.

For some embodiments, the gene pair, the first and second genes, incorporated to the cassettes can be ID11, tHMG1, ERG 8, ERG10, ERG12, ERG13, ERG19 and ERG 20 to at least partially build one or more additional mevalonate pathway inside the transformed yeast by the disclosed method. In other embodiments, the cassette may carry a pair of genes applicable for isobutanol production. The genes associated to isobutanol synthesis are ARO10, ADH7, ILV2, ILV3 and ILV5.

ERG10: acetyl-CoA C-acetyltransferase (EC: 2.3.1.9)
ERG13: hydroxymethylglutaryl-CoA synthase (EC: 2.3.3.10)
tHMG1: truncated version of hydroxymethylglutaryl-CoA reductase
(EC: 1.1.1.34)
ERG12: mevalonate kinase (EC: 2.7.1.36)
ERG8: phosphomevalonate kinase (EC: 2.7.4.2)
ERG19: diphosphomevalonate decarboxylase (EC: 4.1.1.33)
IDI1: isopentenyl-diphosphate delta-isomerase (EC: 5.3.3.2)
ERG20: bifunctional (2E,6E)-farnesyl diphosphate synthase/
dimethylallyltranstransferase (EC: 2.5.1.10 2.5.1.1)
ILV2: acetolactate synthase (EC: 2.2.1.6)
ILV5: ketol-acid reductoisomerase (EC: 1.1.1.86)
ILV3: dihydroxy-acid dehydratase (EC: 4.2.1.9)
ARO10: Transaminated amino acid decarboxylase (EC: 4.1.1.43)
ADH7: NADP-dependent alcohol dehydrogenase 7 (EC: 1.1.1.2)

Further, the cassette employed in the disclosed method may also contain an element to signal the encoded polypeptides or proteins to be transported to mitochondria of the transformed yeasts thereby utilizing resources or precursors found in the mitochondria to biosynthesize the compound of interest. Preferably, the mentioned element is mitochondrial localization or targeting signaling sequence fused to the gene constructed to the cassettes. The mitochondrial targeting sequence applicable for the cassette of the disclosed method can be from COX4, COQ3, etc.

Particularly, the disclosed method exploits δ-integration coupled with antibiotic selection to rapidly assemble large biochemical pathways in budding yeast. The disclosed method also offers several advantages compared to other widely used approaches for the rapid construction of biochemical pathways (9, 38). Firstly, other developed methodologies, like DNA assembler, cannot be used to adjust number of gene copy for modulating enzyme expression level, while it is expected that increased duplication of genes involved in the rate limiting steps should improve the efficiency of the assembled pathway. Secondly, since yeast does not readily express polycistronic genes, different promoter and terminator flanking sequences are required for the functional expression of each integrated gene in the established DNA assembler method. It is a must for the DNA assembler method to avoid the reuse of identical or similar regulatory elements when assembling long biochemical pathway, as any internal recombination will result in incomplete biochemical pathways. On the contrary, only one pair of well-characterized promoter and terminator is needed for the disclosed method. As proven in the below examples, the disclosed method is capable of constructing the five-gene isobutanol pathway and the eight-gene mevalonate pathway using the disclosed δ-integration platform with only one bidirectional promoter employed. The present disclosure eliminates the requirement of meticulous selection on sets of appropriate promoter and terminator sequences prior to cassette assembly. Since the present described method only requires simple plasmid preparation and one-step yeast transformation to assemble the entire biochemical pathway, the disclosed method represents a powerful tool in the construction of large biochemical pathways for synthetic biology, metabolic engineering and pathway engineering.

Another aspect of the present disclosure refers to a method of transforming yeast by way of genome integration and identifying the transformed yeasts with the desired trait imparted through putting selection pressure towards the cultured yeasts. Preferably, the selection pressure is an external factor that its influence or pressure towards the transformed yeast can be conveniently adjusted or regulated by a practitioner of disclosed method. More particularly, the method comprises the steps of transporting a plurality of linear expression cassettes into yeasts to produce transformed yeasts with at least one copy of the cassette integrated into the genome that the linear expression cassette has a dominant selection marker against an antibiotic; and selecting the transformed yeast with a minimal number of expression cassette integrated based upon resistance capacity of the transformed yeasts against the antibiotic of a predetermined concentration by growing the transformed yeast in the presence of the antibiotic of the predetermined concentration. Preferably, the resistance capacity of the transformed yeast is substantially in positive correlation with the number of expression cassette integrated into the genome of the transformed yeasts. In accordance with several embodiments, the antibiotic against by the selection marker is phleomycin and the predetermined concentration is at least 40 μg/mL. Furthermore, the transporting step or the transformation of the yeasts is performed using electroporation, lithium acetate/poly(ethylene glycol), or other approaches, with or without known modification, in a plurality of embodiment of the mentioned method.

The following example is intended to further illustrate the invention, without any intent for the invention to be limited to the specific embodiments described therein.

### Example 1

Escherichia coli strain DH5a or TOP10 were used for routine transformation and strains were cultivated at 37° C. in Luria-Bertani (LB) medium. S. cerevisiae BY4742 strain (MATα; his3Δ, 1; leu2Δ, 0; lys2Δ, 0; ura3Δ, 0) and CEN.PK2-1C were obtained from EUROSCARF. S. cerevisiae BY4742 strain was used as the parent strain for all yeast strain constructions. YPD medium was used for culturing parental strains and engineered strains without plasmid. Synthetic complete (SC) media with leucine and/or uracil dropped out where appropriate were used for maintaining engineered strains transformed with plasmids harboring different auxotrophic selection markers. For the induction of genes under the control of galactose inducible promoters, S. cerevisiae strains were grown in galactose as carbon source. Plasmid pUG66, pKT127, pSH47 and pUG73 were all obtained from EUROSCARF. Plasmid pUC18 and pYES2 were obtained from Invitrogen, Singapore. pESC-URA was purchased from Life Technologies, Singapore. Plasmid pRS425ADS harboring the codon optimized amorpha-4,11-diene synthase gene from Artemisia annua (6) was a gift from Prof. Jay Keasling at University of California, Berkeley. All the chemicals used in the present study were purchased from Sigma-Aldrich (St. Louis, Mo., USA). Restriction enzymes, Taq polymerase, alkaline phosphatase (CIP) and T4 ligase were obtained from New England Biolabs (Beverly, Mass., USA). iProof HF polymerase and iScript™ Reverse Transcription Supermix were purchased from BioRad (Hercules, Calif., USA). QIAquick Gel Extraction Kit, QIAprep Spin Miniprep Kit and RNeasy Mini Kit were all purchased from QIAGEN (Singapore, SG). FastStart Essential DNA Green Master was purchased from Roche. Oligonucleotides were synthesized by integrated DNA technologies (Singapore, SG). DNA sequencing service was provided by 1st BASE (Singapore, SG).

### Example 2

Oligonucleotides used for plasmid constructions were listed in Table 1. To create the platform for multiple-genome integration into yeast chromosomes, the new δ-integration platform was constructed as follows. The δ sequence was amplified from genomic DNA of BY4742, cut with HindIII/EcoRI and inserted into pUC18, to yield pδ-BLANK. The δ sequence used in the present study was provided in Table 2. The plasmid pδ-BLANK was cut with XhoI and dephosphorylated by CIP, inserted with the dominant selection marker amplified from pUG66 using primer pair F_BLE_SS and R_BLE_SalI to create pδBLE (Overlapping PCR was performed to remove the internal SalI restriction site of selection marker). Next, the dual promoter pGAL1/10 cassette was amplified from a modified version of pESC-URA (with an additional SalI introduced between the original SacI and BglII) with primer pair F_tADH1_SphI and R_tCYC1_SphI, digested with SphI and inserted into pδBLE at the same site, to yield pδBLE2.0. Next, mitochondrial targeting sequence from subunit IV of yeast cytochrome c oxidase (encoded by COX4) (26) was amplified from BY4742 genomic DNA using primer pair F_NCOX4_BamHI and R_NCOX4_BS, and inserted into pδBLE2.0 cut with BglII and SalI, to yield pδBLE2.1. For the initial test of genome integration platform, EGFP gene was amplified from plasmid pKT127, cut with BamHI/XhoI and inserted into pδBLE2.1 cut with the same enzyme pair, to yield pδBLE2.1-EGFP.

TABLE 1
Oligonucleotides used for platform and/or cassettes construction

| Listing no. | Name | Discription |
|---|---|---|
| Seq. ID No. 1 | F_Delta_Hind | ACCCCAAGCTTTGTTGGAATAAAAATCCACTATC |
| | III | |
| Seq. ID No. 2 | R_Delta_EcoRI | GGAATTCATGGGGGTTCTCTGGAACAG |
| Seq. ID No. 3 | F_Delta_integ | TGTTGGAATAAAAATCCACTATC |
| Seq. ID No. 4 | R_Delta_integ | ATGGGGGTTCTCTGGAACAG |
| Seq. ID No. 5 | F_BLE_SS | ACACGCGTCGACGCATGCATAACTTCGTATAATGTATGCT |
| | | ATACGAAGTTATCCTCGTCCCCGCCGGGTC |
| Seq. ID No. 6 | R_BLE_SalI | ACACGCGTCGACATAACTTCGTATAGCATACATTATACGA |
| | | AGTTATCGAGAGCTCGTTTTCGAC |
| Seq. ID No. 7 | F_pGAL1_SphI | AGAGAGCATGCACGGATTAGAAGCCGCCGAG |
| Seq. ID No. 8 | R_tCYC1_SphI | AGAGAGCATGCCTTCGAGCGTCCCAAAACCTTC |
| Seq. ID No. 9 | F_pGAL1Screen | CGTCAAGGAGAAAAAACCCC |
| Seq. ID No. 10 | R_tCYC1Screen | CTTTTCGGTTAGAGCGGATC |
| Seq. ID No. 11 | F_ERG10_BamHI | CGGGATCCAAAACAATGTCTCAGAACGTTTACATTG |
| Seq. ID No. 12 | R_ERG10_XhoI | ACACGCTCGAGTCATATCTTTTCAATGACAATAG |
| Seq. ID No. 13 | F_ERG13_BamHI | CGGGATCCAAAACAATGAAACTCTCAACTAAACTTTG |
| Seq. ID No. 14 | R_ERG13_XhoI | ACACGCTCGAGTTATTTTTTAACATCGTAAGATC |
| Seq. ID No. 15 | F_tHMG1_BamHI | CGGGATCCAAAACAATGGCTGCAGACCAATTGGTG |
| Seq. ID No. 16 | R_tHMG1_XhoI | ACACGCTCGAGTTAGGATTTAATGCAGGTGAC |
| Seq. ID No. 17 | F_ERG12_BamHI | CGGGATCCAAAACAATGTCATTACCGTTCTTAAC |
| Seq. ID No. 18 | R_ERG12_OE | GCTAACAACGGGTCCAAAAGAC |
| Seq. ID No. 19 | F_ERG12_OE | GTCTTTTGGACCCGTTGTTAGC |
| Seq. ID No. 20 | R_ERG12_XhoI | ACACGCTCGAGTTATGAAGTCCATGGTAAATTC |
| Seq. ID No. 21 | F_ERG8_BamHI | CGGGATCCAAAACAATGTCAGAGTTGAGAGCC |
| Seq. ID No. 22 | R_ERG8_XhoI | ACACGCTCGAGTTATTTATCAAGATAAGTTTCCG |
| Seq. ID No. 23 | F_ERG19_BamHI | CGGGATCCAAAACAATGACCGTTTACACAGCATC |
| Seq. ID No. 24 | R_ERG19_XhoI | ACACGCTCGAGTTATTCCTTTGGTAGACCAG |
| Seq. ID No. 25 | F_IDI1_BamHI | CGGGATCCAAAACAATGACTGCCGACAACAATAG |
| Seq. ID No. 26 | R_IDI1_XhoI | ACACGCTCGAGTTATAGCATTCTATGAATTTG |
| Seq. ID No. 27 | F_ERG20_BamHI | CGGGATCCAAAACAATGGCTTCAGAAAAAGAAATTAG |
| Seq. ID No. 28 | R_ERG20_XhoI | ACACGCTCGAGCTATTTGCTTCTCTTGTAAAC |

TABLE 2
The delta sequence used for the present study

| Listing no | Sequence detail |
|---|---|
| Seq. ID No. 29 | AAGCTTTGTTGGAATAGAAATCAACTATCATCTACTAACTAGTATTTACATTA |
| for | CTAGTATATTATCATATACGGTGTTAGAAGATGACGCAAATGATGAGAAATA |
| Delta sequence | GTCATCTAAATTAGTGGAAGCTGAAACGCAAGGATTGATAATGTAATAGGAT |
| (Flanked with | CAATGAATATAAACATATAAAACGGAATGAGGAATAATCGTAATATTAGTAT |
| HindIII and | GTAGAAATATAGATTCCATTTTGAGGATTCCTATATCCTCGAGGAGAACTTCT |
| EcoRI) | AGTATATTCTGTATACCTAATATTATAGCCTTTATCAACAATGGAATCCCAAC |
| | AATTATCTCAACATTCACCCATTTCTCATGGTAGCGCCTGTGCTTCGGTTACT |
| | TCTAAGGAAGTCCACACAAATCAAGATCCGTTAGACGTTTCAGCTTCCAAAA |
| | CAGAAGAATGTGAGAAGGCTTCCACTAAGGCTAACTCTCAACAGACAACAA |
| | CACCTGCTTCATCAGCTGTTCCAGAGAACCCCCATGAATTC |

### Example 3

For constructing mitochondrion-based expression of isobutanol pathway, intermediate plasmids were constructed as follows. To target KDC and ADH for the expression into mitochondria, ARO10 and ADH7 were amplified from BY4742 genomic DNA, and inserted into BglII/SalI sites of pδBLE2.1. The subsequent constructed plasmids were designated as pδBLE2.1-ARO10 and pδBLE2.1-ADH7. Genes involved in valine biosynthesis pathway, namely, ILV2, ILV5 and ILV3, were also amplified from the genomic DNA of S. cerevisiae and inserted into BamHI/XhoI site of pδBLE2.1, to yield pδBLE2.1-ILV2, pδBLE2.1-ILV5 and pδBLE2.1-ILV3, respectively. To this end, all isobutanol pathway genes were put under the control of strong galactose inducible promoters (as shown in Table 3). These plasmids were next served as templates for the subsequent PCR amplification of genome integration cassettes to achieve combinatorial genome integration of isobutanol pathway genes. Specifically, primer pair F_Delta_Integ/R_pGAL1/10_Integ was used to amplify upstream module and F_pGAL1/10_Integ/R_Delta_Integ was used for the amplification of downstream module, so that the entire isobutanol pathway can be combinatorially integrated into δ-sites of Ty elements.

TABLE 3
List of plasmids

| Plasmid name | Description |
|---|---|
| pUC18 | |
| pYES2 | |
| pUG66 | |
| YB/I | YEplac 195::TDH3p-crtYB-CYC1t; |
| | TDH3p-crtI-CYC1t |
| pRS425ADS | pRS425::GAL1p-ADS-CYC1t |
| pδ-BLANK | pUC18 derivative containing Delta sequence |
| pδBLE | pδ-BLANK derivative with BLE selection marker |
| pδBLE1.1 | pδBLE derivative with insersion of GAL1p-CYC1t |
| pδBLE1.1-ERG10 | pδBLE1.1::GAL1p-ERG10-CYC1t |
| pδBLE1.1-ERG13 | pδBLE1.1::GAL1p-ERG13-CYC1t |
| pδBLE1.1-tHMG1 | pδBLE1.1::GAL1p-tHMG1-CYC1t |
| pδBLE1.1-ERG12 | pδBLE1.1::GAL1p-ERG12-CYC1t |
| pδBLE1.1-ERG8 | pδBLE1.1::GAL1p-ERG8-CYC1t |
| pδBLE1.1-ERG19 | pδBLE1.1::GAL1p-ERG19-CYC1t |
| pδBLE1.1-IDI1 | pδBLE1.1::GAL1p-IDI1-CYC1t |
| pδBLE1.1-ERG20 | pδBLE1.1::GAL1p-ERG20-CYC1t |

For library construction, electroporation was performed as follows. 10 mL YPD medium was inoculated with overnight S. cerevisiae BY4742 culture to an initial OD600 of 0.3, Yeast cells were harvested by centrifugation at 4° C., 3000 rpm for 5 min after 4-5 h when OD600 reached 1.2. The cell pellet was washed twice with 10 mL ice-cold Milli-Q water, followed by centrifugation to collect cells. Next, cells were washed with 1 mL ice-cold 1 M sorbitol, pelleted by centrifuge and finally re-suspended in ice-cold sorbitol to a final volume of 50 μL.

Subsequently, 50 μL of yeast cells together with approximately 10 μg mixture of equimolar individual integration cassette was electroporated in a 0.2 cm cuvette at 1.6 kV. After electroporation, cells were immediately mixed with 3 mL pre-warmed YPD medium and shaken 2-3 h on a rotary shaker to recover cells. Following that, cells were collected by centrifugation at 3000 rpm for 5 min on a centrifuge, washed and re-suspended in ddH2O. For the construction of isobutanol pathway library, appropriate amount of cells was plated on YPD plate supplemented with 40 μg/mL, 80 μg/mL and 160 μg/mL of phleomycin (InvivoGen, San Diego, USA). Overall, three libraries for isobutanol biosynthesis were constructed with cells adapted to different concentrations of phleomycin.

For the characterization of isobutanol production in the engineered yeast strains, small-scale study was carried out in 14 mL conical tubes. Specifically, conical tubes containing 2 mL SC medium (3.8% galactose and 0.2% glucose) with valine dropped out were inoculated to an initial OD600 of 0.05 with overnight cultures. Alcohol levels were measured after 4 days of cultivation. 100 μL of cell culture was aliquoted for measuring OD600 by microplate reader (Synergy H1, BioTek, USA). Alcohol compounds were isolated by solvent extraction. 500 μL of supernatant of culture broth after centrifugation was extracted with 1 mL GC standard grade toluene spiked with n-butanol. 900 uL of organic phase was next diluted with 900 uL of toluene and subjected to GC-MS analysis. For GC-MS analysis, 1 μL of diluted sample was injected into Shimadzu QP2010Ultra system equipped with a DB5 ms column (30 m×250 μm×0.25 μm thickness) (Agilent Technologies, USA). Split ratio was set with 15:1. Helium (ultra purity) was used as carrier gas at a flow rate 1.0 mL/min. GC oven temperature was initially held at 40° C. for 2 min, ramped with a gradient of 5° C./min until 45° C. and held for 4 min. And then it was raised with a gradient 15° C./min until 230° C. and held for 4 min. The injector and detector were maintained at 225° C. Scan mode was used to detect mass range 40-120 m/z. For the quantitation of isobutanol produced by engineered strains, authentic alcohol compound from Sigma-Aldrich was used for plotting standard curve. To correct for differences during sample preparation, the internal spiked n-butanol was used for normalizing the values.

Combinatorial assembly of mitochondrion-based isobutanol pathway into yeast chromosomes For synthetic biology applications, it often requires the transfer of the entire biosynthetic pathways from native hosts such as plants and mammals into microbes to achieve high-level production of value-added chemicals through large-scale fermentation processes. Thus inventors of the present disclosure decided to investigate whether it is possible to harness the δ-sites of retrotransposons (Ty) elements (14, 15) coupled with antibiotic selection for the rapid construction of large biochemical pathways into yeast chromosomes (FIG. 1). The idea was originated from the approach called chemically induced chromosomal evolution (CIChE), which has been used to evolve E. coli chromosome for gene duplications using antibiotic of chloramphenicol (20). In the present study, the present disclosure chose to use dominant antibiotic selection marker against phleomycin (a glycopeptide antibiotic of the bleomycin family that can introduce DNA breaks), as very low concentration of phleomycin (10 μg/mL) is sufficient for the selection of gene deletion events (21).

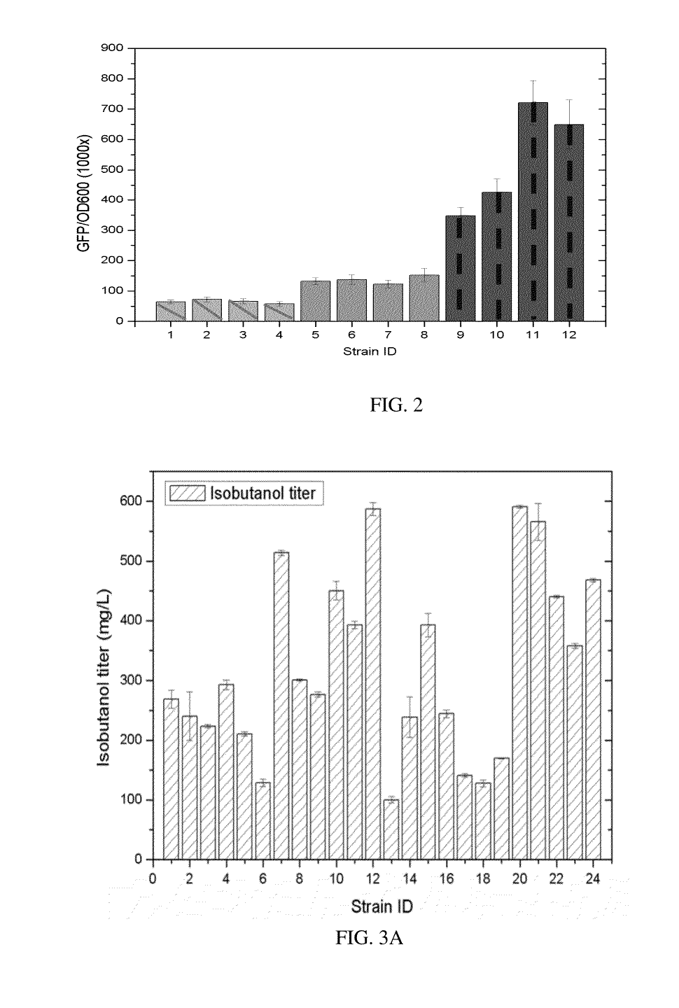

Before exploiting combinatorial integration of biochemical pathways into yeast chromosomes, the 6-integration platform was first tested for integrating a reporter gene encoding enhanced green fluorescent protein (EGFP) to examine whether it is possible to achieve various degrees of integration events by simply modulating the concentration of antibiotics. As can be seen in FIG. 2, engineered strains obtained from library supplemented with higher concentration of antibiotics showed significantly higher levels of fluorescent intensities over engineered strains from library with lower selection pressure, which confirmed that the feasibility of modulating genome integration events by simply selecting with different concentrations of antibiotics.

Previously, the isobutanol pathway has been constructed in their natural compartments by overexpressing only some of pathway genes to increase isobutanol production (22-24). Simple overexpression of the isobutanol pathway genes in their natural compartments to improve isobutanol titer is not only limited by the transport of intermediates from mitochondrion to cytoplasm, but also limited by competing pathways. In contrast, eliminating bottleneck posed by the transport of intermediates across membranes showed great promise for high-level production of isobutanol, to a titer around 650 mg/L by either re-localization of the upstream isobutanol biosynthetic pathway to cytoplasm (25) or sequestration of downstream pathway into mitochondria (16). The present disclosure aimed to further optimize mitochondrion-based isobutanol biosynthetic pathway by eliminating plasmid-based overexpression system and combinatorially balancing of pathway gene expression levels. To achieve mitochondrion-based isobutanol production, α-ketoacid decarboxylase (encoded by ARO10) and alcohol dehydrogenase (encoded by ADH7) were fused with the N-terminal mitochondrial localization signal from subunit IV of the yeast cytochrome c oxidase (encoded by COX4) (26), in a similar way as carried out by previous study (16). Here, the experiment proceeded to construct isobutanol-producing mutant libraries, and sought to use three different concentrations of antibiotics (40 μg/mL, 80 μg/mL and 160 μg/mL of phleomycin) for different degrees of genome integration events. To examine whether isobutanol pathway genes have been successfully integrated into yeast chromosomes, universal primer pairs F_GAL10Scr/R_GAL10Scr and F_GAL1Scr/R_GAL1Scr were used to verify genome integration events. As can be seen in Table 4, PCR results confirmed there were successful genome integration events for isobutanol pathway genes. And there was a clear trend of more isobutanol pathway genes being detected for engineered strains from library treated with high concentration of antibiotics. Interestingly, when the genome integration cassettes were partitioned into upstream and downstream modules, there was biased genome integration as genes with smaller size such as ILV5 and ADH7 (gene size around 1.2 kb) were integrated into yeast chromosomes for all engineered strains (Table 4). In contrast, the chance for ILV2, ILV3 and ARO10 with size around 2 kb to be integrated into yeast chromosomes was lower. The entire isobutanol biosynthesis pathway was integrated into yeast chromosomes for the majority of engineered strains from library treated with 160 μg/mL of phleomycin. Based on these findings, biased integration should be considered for the future library construction and one possible solution would be adjusting DNA input in the mixture during electroporation.

TABLE 4
PCR verification of genome integration events for
mutant strains from the isobutanol pathway library

| | Library (L) ^{a} | | | | | | Library (M) ^{b} | | | | | | Library (H) ^{c} | | | | | |
|---|---|---|---|---|---|---|---|---|---|---|---|---|---|---|---|---|---|---|
| | 1 | 2 | 3 | 4 | 5 | 6 | 1 | 2 | 3 | 4 | 5 | 6 | 1 | 2 | 3 | 4 | 5 | 6 |
| ILV2 | − | − | + | − | − | − | − | − | − | − | + | − | + | + | − | + | + | + |
| (2.1 kb) | | | | | | | | | | | | | | | | | | |
| ILV3 | − | + | − | − | − | + | + | + | + | + | − | + | + | + | + | + | + | + |
| (1.8 kb) | | | | | | | | | | | | | | | | | | |
| ILV5 | + | + | + | + | + | + | + | + | + | + | + | + | + | + | + | + | + | + |
| (1.2 kb) | | | | | | | | | | | | | | | | | | |
| ARO10 | − | − | − | − | − | − | − | − | − | − | + | − | + | + | + | + | + | − |
| (2.0 kb) | | | | | | | | | | | | | | | | | | |
| ADH7 | + | + | + | + | + | + | + | + | + | + | + | + | + | + | + | + | + | + |
| (1.2 kb) | | | | | | | | | | | | | | | | | | |

^{a} Library (L) was mutant strains from library supplemented with 40 μg/mL of phleomycin.
^{b} Library (M) was mutant strains from library supplemented with 80 μg/mL of phleomycin.
^{c} Library (H) was mutant strains from library supplemented with 160 μg/mL of phleomycin.

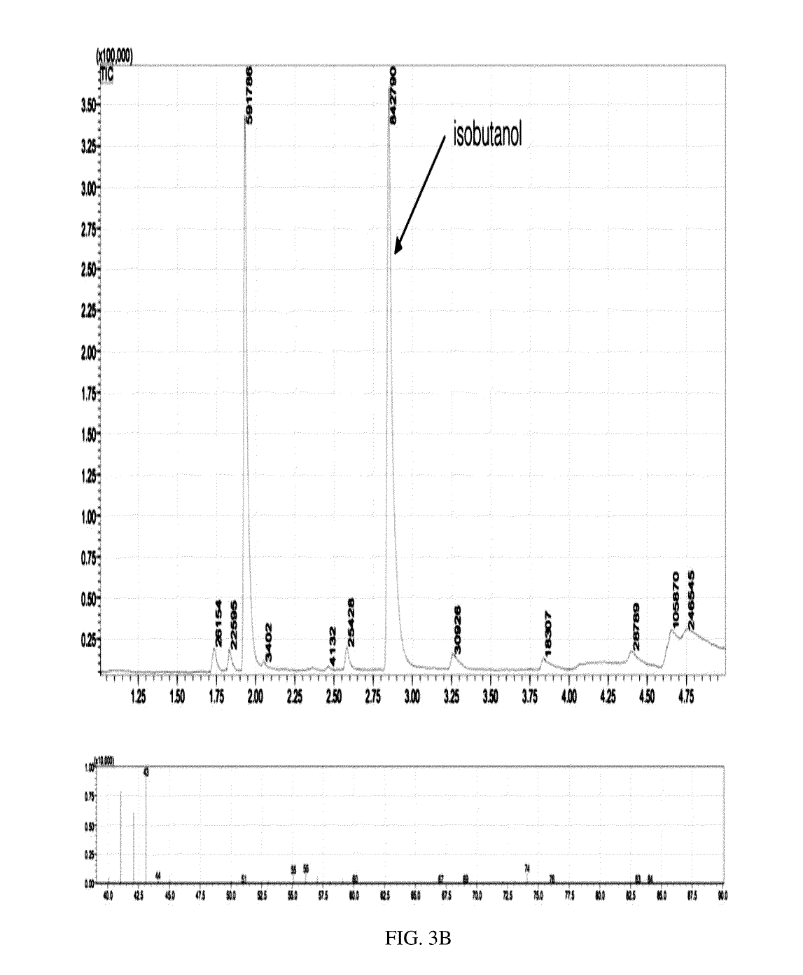

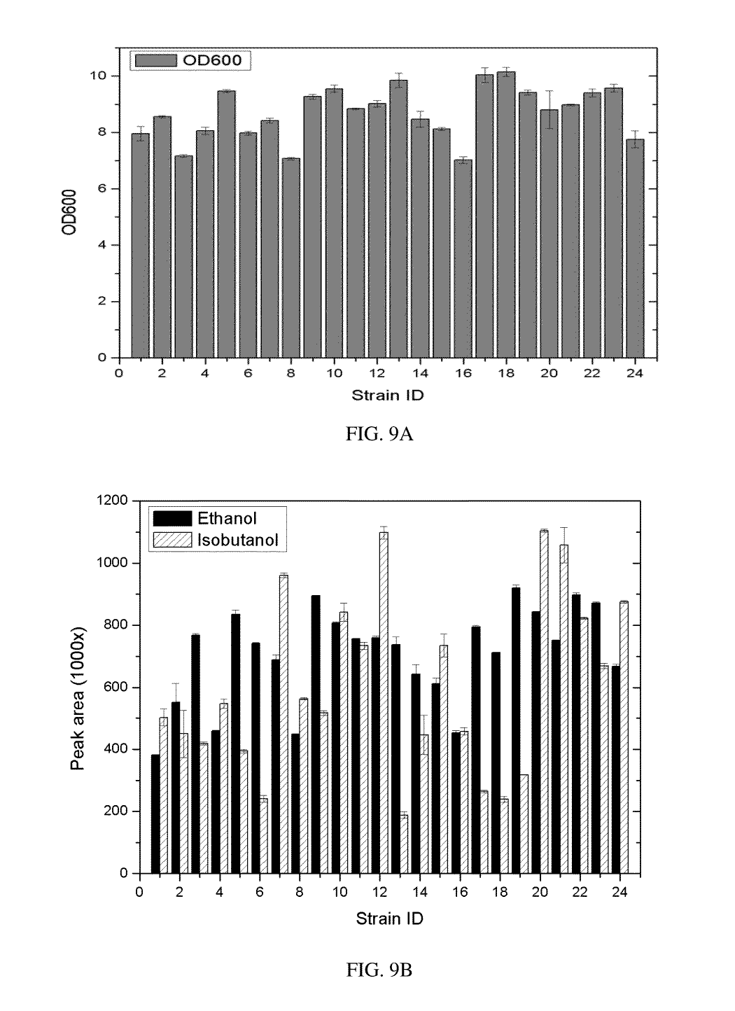

As majority of engineered strains from the library supplemented with 160 μg/mL of phleomycin appeared to have the entire mitochondrion-based isobutanol pathway, the present disclosure thus randomly tested twenty-four engineered strains from this library to determine whether these engineered strains could yield high levels of isobutanol. As can be seen from FIG. 3A, distinct levels of isobutanol were observed for engineered strains after 4 days cultivation. There were trace amounts of C5 alcohols such as isopentanol and 2-methyl-1-butanol detected during GC-MS analysis (FIG. 3B). The variations of isobutanol titers in engineered strains suggested that there should be beneficial combinations of integration events for higher-level production of isobutanol. It was surprising to find out that poor cell densities did not correlate with isobutanol levels (FIG. 9). For example, engineered strain M8 and M16 with poor growth did not produce the highest levels of isobutanol; however, the best producers such as M12 and M20 showed only minor growth impairment when compared to the parental strain without genetic modification (Data not shown). However, there was good correlation of ethanol levels with cell densities as shown from FIG. 9. Since no detectable accumulation of intermediates such as isobutanal was observed during GC-MS analysis for all engineered strains, it suggested that the enzymatic activity of alcohol dehydrogenase encoded by ADH7 was sufficient to convert isobutanal into isobutanol.

Among twenty-four engineered strains, the best variant (M20) produced approximately 600 mg/L of isobutanol (FIG. 3A), which was impressive as only one round of combinatorial genome integration was performed. In comparison, the reference strain without genetic modification only produced approximately 6 mg/L of isobutanol. Here, the resulted strains did produce isobutanol more efficiently than plasmid-based expression system as significantly less sugar was consumed (16), which confirmed that eliminating plasmid burden and combinatorially balancing metabolic flux could generate yeast strains with better performance. However, as isobutanol was reported to reach 20 g/L in the engineered E. coli (27-29), there is still a significant room for improvement in yeast to achieve industrial-scale production of isobutanol to be considered as gasoline substitutes.

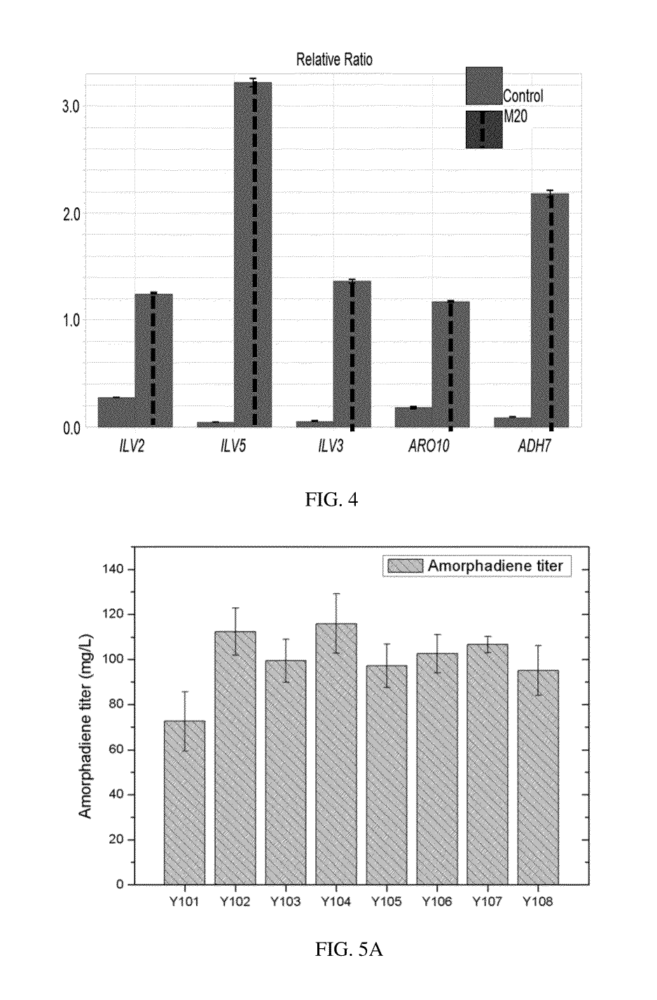

To confirm the improvement of isobutanol titer in the engineered strains was attributed to the overexpression of isobutanol pathway genes, inventors of the present disclosure decided to verify of gene expression profile of M20 by carrying out quantitative real-time reverse-transcription PCR (qRT-PCR) studies. To correct for differences in the amounts of starting materials, ACT1 was chosen as a reference housekeeping gene and the results were presented as ratios of gene expression between the target gene (gene of interest) and the reference gene, ACT1 (30). As can be seen from FIG. 4, gene expression levels of all isobutanol pathway genes in M20 were significantly higher when compared to those of the parental strain, which confirmed all isobutanol pathway genes were successfully integrated into yeast chromosomes for overexpression. Among them, ILV5 and ADH7 showed the highest abundances of mRNA levels, presumably due to multiple copies of these genes into yeast chromosomes.

### Example 4

All eight genes involved in mevalonate pathway were amplified from genomic DNA of S. cerevisiae BY4742 using primers listed in Table 1. Overlapping PCR was used to remove BamHI site from ERG12 gene. All PCR products were digested with BamHI/XhoI. The digested products of ERG13, ERG12, ERG19 and ID11 were inserted into pδBLE2.0 cut with BglII/SalI, to yield a series of intermediate plasmids. Next, ERG10, tHMG1, ERG8 and ERG20 were inserted into intermediate plasmids cut with BamHI/XhoI. Subsequently constructed plasmids were named as pδBLE2.0-ERG13/ERG10, pδBLE2.0-ERG12/tHMG1, pδBLE2.0-ERG19/ERG8 and pδBLE2.0-ID11/ERG20 (as listed in Table 3). To this end, all the mevalonate pathway genes were put under the control of galactose inducible strong promoter. Next, these plasmids were served as template and primer pair F_Delta_Integ/R_Delta_Integ was used for the subsequent PCR amplification of genome integration cassettes.

For library construction, electroporation was performed as follows. 10 mL YPD medium was inoculated with overnight S. cerevisiae BY4742 culture to an initial OD600 of 0.3, Yeast cells were harvested by centrifugation at 4° C., 3000 rpm for 5 min after 4-5 h when OD600 reached 1.2. The cell pellet was washed twice with 10 mL ice-cold Milli-Q water, followed by centrifugation to collect cells. Next, cells were washed with 1 mL ice-cold 1 M sorbitol, pelleted by centrifuge and finally re-suspended in ice-cold sorbitol to a final volume of 50 μL. Subsequently, 50 μL of yeast cells together with approximately 10 μg mixture of equimolar individual integration cassette was electroporated in a 0.2 cm cuvette at 1.6 kV. After electroporation, cells were immediately mixed with 3 mL pre-warmed YPD medium and shaken 2-3 h on a rotary shaker to recover cells. Following that, cells were collected by centrifugation at 3000 rpm for 5 min on a centrifuge, washed and re-suspended in ddH2O. For the construction of mevalonate pathway library, cells were directly spotted on YPD agar plate supplemented with even higher concentration of phleomycin to a final concentration of 240 μg/mL to achieve integration of the entire mevalonate pathway into yeast chromosomes.

Fresh overnight cell culture was inoculated into 2 mL SC media to an initial OD600 of 0.05. Cells were cultured on a rotary shaker for 12 h and harvested during the exponential growth phase. Approximately 1×10⁷ cells were collected by centrifugation and washed twice with phosphate saline buffer. Next, the total RNA was extracted using RNeasy Mini Kit (QIAGEN, Germany). During RNA extraction, genomic DNA contamination was eliminated by in-column digestion with DNase I (QIAGEN, Germany). The concentration of eluted RNA solution was quantified by measuring the absorbance at 260 nm. Approximately 500 ng of RNA was converted to cDNA using iScript™ Reverse Transcription Supermix from Biorad. Oligonucleotides for qRT-PCR studies of pathway genes and actin (ACT), internal reference gene) were designed using the ProbeFinder (https://www.roche-applied-science.com), and oligonucleotides used for qRT-PCR experiments were listed in Table 5. qRT-PCR analysis was performed using LightCycler 96 real-time machine with FastStart Essential DNA Green Master according to the manufacturer's instructions. Each 20 μL reaction contained 50 ng of total cDNA, 10 μL FastStart Essential DNA Green Master, 0.5 μM of each primer. Thermal cycling conditions were set as follows: pre-incubation, 1 cycle of 95° C. for 10 min; amplification, 45 cycles of 95° C. for 10 s, 57° C. for 10 s and 72° C. for 10 s. To correct for differences in the amounts of starting materials, ACT1 was chosen as a reference housekeeping gene. The results were presented as ratios of gene expression between the target gene (gene of interest) and the reference gene, ACT1 (30). All assays were performed in triplicate, and the reaction without reverse transcriptase was used as a negative control.

TABLE 5
List of primers used for qPCR.

| | | Length | Tm | GC | |
|---|---|---|---|---|---|
| Listing no. | Name | (nt) | (° C.) | (%) | Discription |
| Seq ID. No. 30 | F_ACT1_q | 18 | 59 | 56 | tccgtctggattggtggt |
| Seq ID. No. 31 | R_ACT1_q | 22 | 59 | 41 | tgagatccacatttgttggaag |
| Seq ID. No. 32 | F_ERG10_q | 20 | 59 | 55 | ccagggttctctatcctcca |
| Seq ID. No. 33 | R_ERG10_q | 19 | 59 | 47 | aggcgccttttaaagcaac |
| Seq ID. No. 34 | F_ERG13_q | 20 | 59 | 45 | atcaccgaaactccaaagga |
| Seq ID. No. 35 | R_ERG13_q | 21 | 60 | 38 | caaatgggcattttctctcaa |
| Seq ID. No. 36 | F_tHMG1_q | 20 | 59 | 50 | tgccatccatcgaagtaggt |
| Seq ID. No. 37 | R_tHMG1_q | 20 | 60 | 50 | catggcaccttgtggttcta |
| Seq ID. No. 38 | F_ERG12_q | 21 | 59 | 48 | gtgtgttggtcaccgagaaat |
| Seq ID. No. 39 | R_ERG12_q | 19 | 60 | 53 | cacattcacccatggcatc |
| Seq ID. No. 40 | F_ERG8_q | 21 | 59 | 52 | aggaggatagcgttaccgaac |
| Seq ID. No. 41 | R_ERG8_q | 21 | 59 | 38 | ttctgtgcgaatgaaaactca |
| Seq ID. No. 42 | F_ERG19_q | 23 | 59 | 39 | ccgaaaataactttcctacagca |
| Seq ID. No. 43 | R_ERG19_q | 19 | 59 | 53 | gagaccaatgcagcaaagc |
| Seq ID. No. 44 | F_IDI1_q | 23 | 59 | 39 | tttccctgatctttggactaaca |
| Seq ID. No. 45 | R_IDI1_q | 21 | 59 | 43 | cccttcaaacctaattcgtca |
| Seq ID. No. 46 | F_ERG20_q | 20 | 60 | 50 | ccggtatcacggatgaaaag |
| Seq ID. No. 47 | R_ERG20_q | 21 | 59 | 43 | ttcacccaatggaatcaagac |

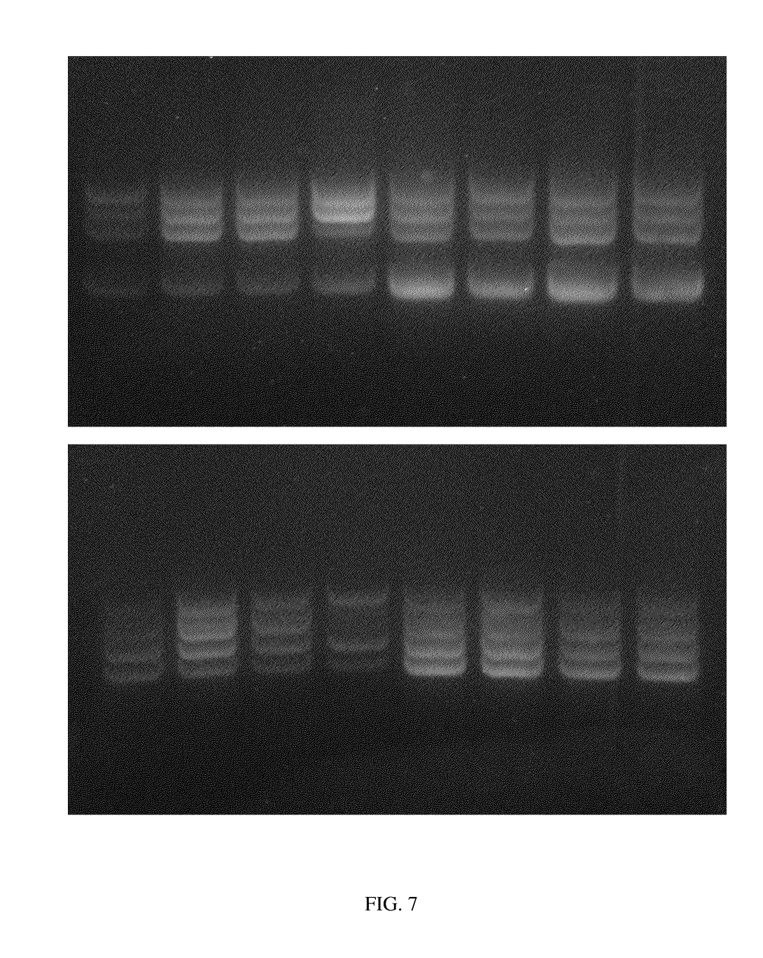

Upon successful assembly of isobutanol pathway, the present disclosure further attempted to reconstruct the eight-gene mevalonate pathway into yeast chromosomes. Here, even higher concentration of antibiotics for selection (240 μg/mL of phleomycin) was used to ensure the entire mevalonate pathway to be integrated into yeast chromosomes. As expected, when eight randomly picked engineered strains were subjected to PCR verification, all engineered strains showed eight-band pattern during PCR verification, which confirmed all mevalonate pathway genes were successfully integrated into yeast chromosomes (FIG. 7).

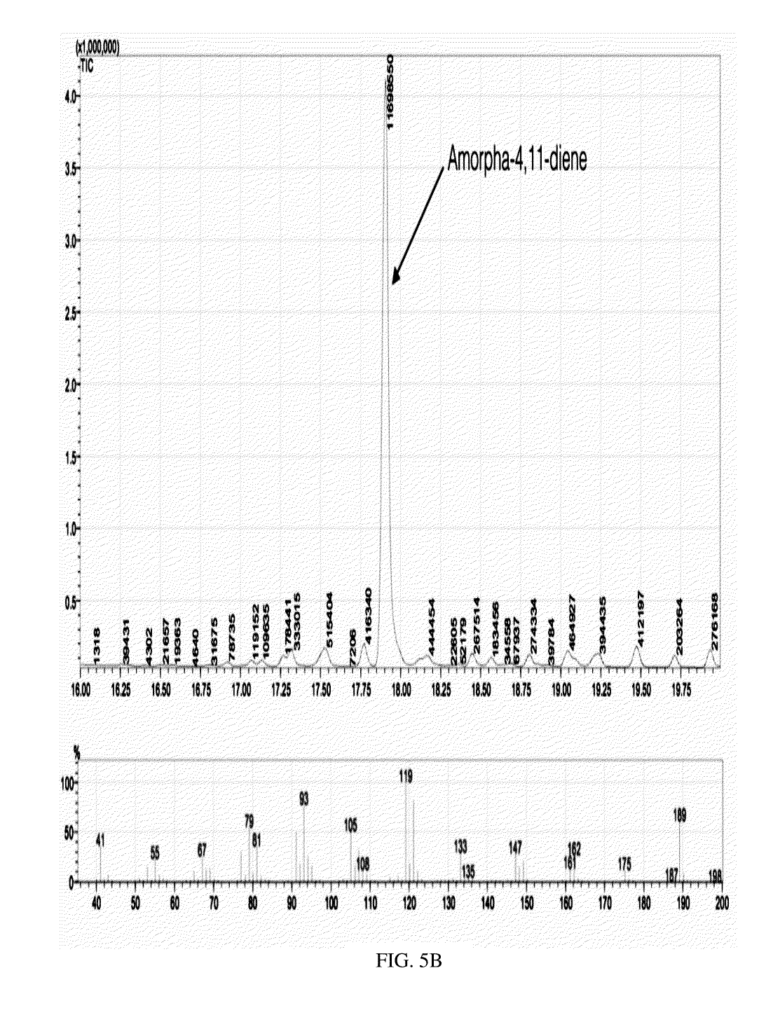

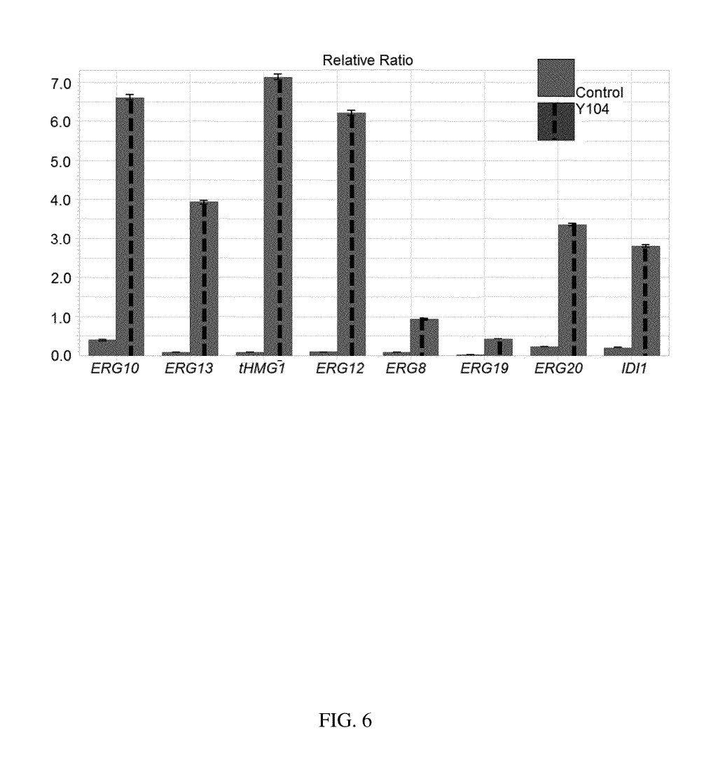

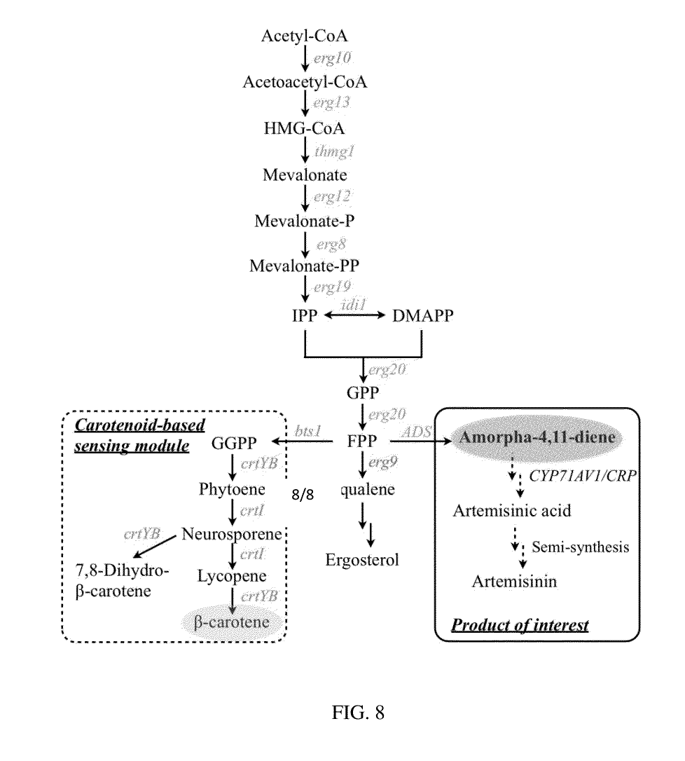

Next, the present disclosure sought to exploit these engineered strains as platform microbes for high-level production of terpenoids, and all eight engineered strains as mentioned above were directly tested for the production of amorpha-4,11-diene, an important precursor for antimalarial drug artimisinin (FIG. 8). As can be seen in FIG. 5, all engineered strains produced high-levels of amorpha-4,11-diene, with titer around 100 mg/L in small-scale shake-tube condition. Among them, the best variant Y104 actually produced around 120 mg/L after 5 days cultivation. Surprisingly, the parental strain transformed with pYES2ADS only produced around 2 mg/L of amorpha-4,11-diene under the same experimental condition, which was lower compared to 4.4 mg/L for the same strain harboring pRS425ADS as reported previously (6). Further qRT-PCR analysis of gene expression levels of Y104 confirmed that mRNA levels of all mevalonate pathway genes were overexpressed (as shown in FIG. 6). Among them, tHMG1 and ERG10 showed the highest expression levels and mRNA abundances of both genes were approximately seven-fold over that of internal control ACT1 (FIG. 6). Interestingly, mRNA abundances for ERG8 and ERG19 were relatively low compared to other mevalonate pathway genes, which probably have beneficial effect on isoprenoid productions as previous investigation showed overexpression of ERG19 would lead to reduced sterol content and the accumulation of diphosphate intermediates led to feedback inhibitions (31). The present disclosure also found all the genes put under control of pGAL1 promoter (ERG10, tHMG1, ERG8 and ERG20) showed slightly higher mRNA abundance over those put under control of pGAL10 promoter (ERG13, ERG12, ERG19 and ID11), which further supported that pGAL1 promoter is generally considered to be stronger over pGAL10 as previously reported (32, 33). Moreover, the possibility of cross talk between different cassettes appears to be negligible, as relative levels for genes in the same cassette were almost consistent. For example, mRNA abundance of tHMG1 under control of pGAL1 over that of ERG12 under control of pGAL10 is similar to that of ERG8 over ERG19 (FIG. 6).

### Example 5

For restricting ERG9 expression in the engineered strain, plasmid pLEU/pCTR3-ERG9 was created as follows. LEU2 selection marker was amplified from pUG73 with primer pair F_LEU_HindIII₁ and R_LEU_EB, cut with HindIII and EcoRI, and inserted into pUC18, to generate intermediate plasmid. Next, the promoter region of pCTR3 was amplified from the genomic DNA of S. cerevisiae CEN.PK2-1C with primer pair F_pCTR3_BamHI and R_pCTR3_EcoRI. The purified PCR product was cut with BamHI and EcoRI, and inserted into the intermediate plasmid to yield pLEU/pCTR3-ERG9. This plasmid was then served as template for the PCR amplification of genome integration cassette using primer pair F_ERG9_Integ/R_ERG9_Integ.

To generate strains with ERG9 under control of copper repressible promoter pCTR3, the preparation of competent cells and electroporation was carried out in a similar way as mentioned in Example 3 and 4 except that cells were mixed together the genome integration cassette amplified from pLEU/pCTR3_ERG9 with primer pair F_ERG9_Integ and R_ERG9_Integ.

Engineered strains were transformed with pYES2ADS or pRS416ADS through conventional lithium acetate approach. Small-scale studies were carried out for amorpha-4,11-diene production. Specifically, 14 mL sterile round bottom tubes containing 2 mL SC medium (1.8% galactose and 0.2% glucose) with uracil dropped out were inoculated to an initial OD600 of 0.05 with overnight cultures. For studying copper-mediated repression of ERG9 to minimize ergosterol biosynthesis, 150 μM of CuSO4 were added into culture media. All tubes were immediately added with 200 μL dodecane after seeding, to perform two phase fermentation and harvest amorpha-4,11-diene. Both amorpha-4,11-diene production and cell density were monitored after 5 days. 100 μL of cell culture was taken for measuring OD600 by microplate reader, and 10 μL dodecane layer was sampled and diluted in 990 μL ethyl acetate for the determination of amorpha-4,11-diene levels by GC-MS. During GC-MS analysis, 1 μL of diluted sample was injected into Shimadzu QP2010Ultra system equipped with a DB5 ms column (Agilent Technologies, USA). Helium was used as a carrier gas at a flow rate of 1.0 mL/min. The oven temperature was first kept constant at 80° C. for 2 min, and then increased to 190° C. at a rate of 5° C./min, and finally increased to 300° C. by 20° C./min. Both the injector and mass detector were set at 250° C. Scan mode was used to detect mass range 40-240 m/z. For the quantitation of amorpha-4,11-diene level, amorpha-4,11-diene was quantitated by GC-MS and FID. Caryophyllene was used for plotting the standard curve, and amorpha-4,11-diene levels in the present study represented as caryophyllene equivalents.

Based on previous findings (6, 34), high-level production of non-native isoprenoid products requires that FPP flux be diverted from the production of sterols to the heterologous metabolic reactions. Down-regulation of ERG9, which encodes squalene synthase (the first committed step after FPP in sterol biosynthesis) by replacing its native promoter with the methionine-repressible MET3 promoter increased amorpha-4,11-diene production an additional 2-fold (6). More recently, copper-regulated CTR3 promoter was used to restrict ERG9 expression by addition of the inexpensive repressor copper sulfate to the medium, and CTR3 promoter for restricting ERG9 expression showed similar effect on improving amorpha-4,11-diene titers (35). Since the native CTR3 promoter in laboratory strain S288C is interrupted by the insertion of a Ty2 element (36), inventors of the present disclosure thus decided to use copper-regulated CTR3 promoter from S. cerevisiae CEN.PK2 strain instead, for restricting ERG9 expression in Y104. The resulted strain with ERG9 under control of copper-repressible promoter pCTR3 was designated as Y104A. To further examine whether modulating ADS expression levels would have profound effect on amorpha-4,11-diene productions, the present disclosure constructed another centromeric plasmid to express ADS gene (pRS416ADS). We found strain Y104A harboring pYES2ADS produced approximately 250 mg/L amorpha-4,11-diene under ERG9 restriction, which confirmed the copper-regulated CTR3 promoter did repress the metabolic flux toward ergosterol biosynthesis and divert the flux to enhanced levels of amporpha-4,11-diene. Interestingly, strain Y104A with pRS416ADS yielded even higher level of amorpha-4,11-diene, to a titer around 500 mg/L under the same condition, which represents the highest reported titer of amorpha-4,11-diene in the laboratory strain S288C. However, when the entire mevalonate pathway was overexpressed in CEN.PK2 strain, it was reported to produce 1200 mg/L of amorpha-4,11-diene under shake-flask condition (37). It is possible to engineer CEN.PK2 strain to utilize glucose as carbon source for high-level production of terpenoids and characterize the product yield under fed-batch condition.

This experiment revealed that engineered strain with centromeric expression of amorpha-4,11-diene synthase was capable of producing approximately 500 mg/L of amorpha-4,11-diene when ERG9 expression was restricted, which represented the best titer of amorpha-4,11-diene in the laboratory strain of S288C.

### Example 6

For the subsequent verification of genome integration events, colonies were first picked up from the library and streaked on phleomycin containing plates to eliminate the false positive strains. Next, universal primer F_pGAL1Scr/R_tCYC1Scr and F_pGAL10Scr/R_tADH1Scr were used for the PCR verification of genome integration of pathway genes. Cells were lysed by 20 mM NaOH for 15 min in 100° C. water bath. PCR program was set as follows: 1 cycle of 95° C. for 5 min; amplification, 30 cycles of 95° C. for 15 s, 50° C. for 30 s and 68° C. for 90 s; 1 cycle of 68° C. for 3 min.

For isobutanol-producing strains, the band size of 2.0 kb and 1.2 kb for the first PCR reaction with primer pair F_pGAL10Scr/R_tADH1Scr corresponded to ARO10 and ADH7, respectively. The band size of 2.1 kb, 1.8 kb and 1.2 kb for the second PCR reaction with primer pair F_pGAL1Scr/R_tCYC1Scr corresponded to ILV2, ILV3 and ILV5, respectively. For mevalonate pathway library, bands with size of 1.5 kb, 1.3 kb, 1.2 kb and 900 bp for the PCR reaction with primer pair F_pGAL10Scr/R_tADH1Scr corresponded to ERG13, ERG12, ERG19 and ID11. Band with size of 1.6 kb, 1.35 kb, 1.2 kb and 1.0 kb for the PCR reaction with F_pGAL1Scr/R_tCYC1Scr corresponded to tHMG1, ERGS, ERG10 and ERG20, respectively.

The present disclosure has successfully demonstrated δ-integration coupled with antibiotic selection for rapid construction of large biochemical pathways into yeast chromosomes. When compared to DNA assembler that has been widely used for the rapid construction of biochemical pathways (9, 38), our approach offers several advantages. For example, DNA assembler cannot be used to adjust gene copy numbers for modulating enzyme expression levels, whereas multiple copies of genes involved in the rate limiting steps are expected to further improve the pathway activity as demonstrated by previous studies (6, 37). Since yeast does not readily express polycistronic genes, different promoter and terminator flanking sequences for individual gene are required for the functional expression in yeast, and it is a must to avoid the reuse of identical or similar regulatory elements when assembling long biochemical pathway using DNA assembler method, as any internal recombination will result in incomplete biochemical pathways. In comparison, only one pair of well-characterized promoter and terminator was used for constructing the five-gene isobutanol pathway and the eight-gene mevalonate pathway using our current δ-integration platform, which eliminates the requirement of meticulously selected sets of appropriate promoter and terminator sequences prior to cassette assembly. And the feature of bidirectional promoter system adopted in the present study may facilitate promoter engineering for tuning enzyme expression levels in a follow-up study to achieve further balanced metabolic flux and improved product titer, in a similar way to the procedure described by previous studies (39, 40). Moreover, when compared to cocktail δ-integration using different auxotrophic selection markers for constructing ratio-optimized cellulolytic enzyme expression in yeast (13), our modified δ-integration with antibiotic selection marker can be easily used for modulating integration events to achieve ratio-optimized expression of pathway genes in one-step fashion, whereas auxotrophic selection marker will typically result in single integration for the cocktail δ-integration system and it will take several rounds of genetic manipulation to introduce more genes into yeast chromosomes. Noteworthy, for the concern of genetic stability of our constructed strains, it would be possible to stabilize the construct by simply deleting Rad51 gene in the engineered strains, in a similar way to RecA deletion carried out by the previous study (20). This is the first report to exploit δ-integration coupled with antibiotic selection to rapid assemble large biochemical pathways in budding yeast. Since this new method only requires simple plasmid preparation and one-step yeast transformation to assemble the entire biochemical pathway, our new approach represents a powerful tool in the construction of large biochemical pathways for synthetic biology, metabolic engineering and pathway engineering studies.

It is to be understood that the present invention may be embodied in other specific forms and is not limited solely to the embodiments described above. However modification and equivalents of the disclosed concepts such as those which readily occur to one skilled in the art are intended to be included within the scope of the claims which are appended thereto.

### REFERENCES

1. Goffeau, A., Barrell, B. G., Bussey, H., Davis, R. W., Dujon, B., Feldmann, H., Galibert, F., Hoheisel, J. D., Jacq, C., Johnston, M., Louis, E. J., Mewes, H. W., Murakami, Y., Philippsen, P., Tettelin, H., and Oliver, S. G. (1996) Life with 6000 genes, Science 274, 546-&.
2. Mumberg, D., Muller, R., and Funk, M. (1994) Regulatable Promoters of Saccharomyces-Cerevisiae—Comparison of Transcriptional Activity and Their Use for Heterologous Expression, Nucleic acids research 22, 5767-5768.
3. Sun, J., Shao, Z. Y., Zhao, H., Nair, N., Wen, F., Xu, J. H., and Zhao, H. M. (2012) Cloning and characterization of a panel of constitutive promoters for applications in pathway engineering in Saccharomyces cerevisiae, Biotechnology and bioengineering 109, 2082-2092.
4. Shao, Z. Y., Zhao, H., and Zhao, H. M. (2009) DNA assembler, an in vivo genetic method for rapid construction of biochemical pathways, Nucleic acids research 37.
5. Flagfeldt, D. B., Siewers, V., Huang, L., and Nielsen, J. (2009) Characterization of chromosomal integration sites for heterologous gene expression in Saccharomyces cerevisiae, Yeast 26, 545-551.
6. Ro, D. K., Paradise, E. M., Ouellet, M., Fisher, K. J., Newman, K. L., Ndungu, J. M., Ho, K. A., Eachus, R. A., Ham, T. S., Kirby, J., Chang, M. C., Withers, S. T., Shiba, Y., Sarpong, R., and Keasling, J. D. (2006) Production of the antimalarial drug precursor artemisinic acid in engineered yeast, Nature 440, 940-943.
7. Pompon, D., Louerat, B., Bronine, A., and Urban, P. (1996) Yeast expression of animal and plant P450s in optimized redox environments, Methods in enzymology 272, 51-64.
8. Eckart, M. R., and Bussineau, C. M. (1996) Quality and authenticity of heterologous proteins synthesized in yeast, Current opinion in biotechnology 7, 525-530.
9. Shao, Z. Y., Luo, Y. Z., and Zhao, H. M. (2011) Rapid characterization and engineering of natural product biosynthetic pathways via DNA assembler, Molecular bioSystems 7, 1056-1059.
10. Oldenburg, K. R., Vo, K. T., Michaelis, S., and Paddon, C. (1997) Recombination-mediated PCR-directed plasmid construction in vivo in yeast, Nucleic acids research 25, 451-452.
11. Gibson, D. G., Glass, J. I., Lartigue, C., Noskov, V. N., Chuang, R. Y., Algire, M. A., Benders, G. A., Montague, M. G., Ma, L., Moodie, M. M., Merryman, C., Vashee, S., Krishnakumar, R., Assad-Garcia, N., Andrews-Pfannkoch, C., Denisova, E. A., Young, L., Qi, Z. Q., Segall-Shapiro, T. H., Calvey, C. H., Parmar, P. P., Hutchison, C. A., 3rd, Smith, H. O., and Venter, J. C. (2010) Creation of a bacterial cell controlled by a chemically synthesized genome, Science 329, 52-56.
12. Anderson, P. R., and Haj-Ahmad, Y. (2003) Counter-selection facilitated plasmid construction by homologous recombination in Saccharomyces cerevisiae, BioTechniques 35, 692.
13. Yamada, R., Taniguchi, N., Tanaka, T., Ogino, C., Fukuda, H., and Kondo, A. (2010) Cocktail delta-integration: a novel method to construct cellulolytic enzyme expression ratio-optimized yeast strains, Microbial cell factories 9, 32.
14. Hauber, J., Nelbock-Hochstetter, P., and Feldmann, H. (1985) Nucleotide sequence and characteristics of a Ty element from yeast, Nucleic acids research 13, 2745-2758.
15. Boeke, J. D., Garfinkel, D. J., Styles, C. A., and Fink, G. R. (1985) Ty elements transpose through an RNA intermediate, Cell 40, 491-500.
16. Avalos, J. L., Fink, G. R., and Stephanopoulos, G. (2013) Compartmentalization of metabolic pathways in yeast mitochondria improves the production of branched-chain alcohols, Nature biotechnology 31, 335-341.
17. Ajikumar, P. K., Xiao, W. H., Tyo, K. E., Wang, Y., Simeon, F., Leonard, E., Mucha, O., Phon, T. H., Pfeifer, B., and Stephanopoulos, G. (2010) Isoprenoid pathway optimization for Taxol precursor overproduction in Escherichia coli, Science 330, 70-74.
18. Jiang, M., Stephanopoulos, G., and Pfeifer, B. A. (2012) Downstream reactions and engineering in the microbially reconstituted pathway for Taxol, Applied microbiology and biotechnology 94, 841-849.
19. Anthony, J. R., Anthony, L. C., Nowroozi, F., Kwon, G., Newman, J. D., and Keasling, J. D. (2009) Optimization of the mevalonate-based isoprenoid biosynthetic pathway in Escherichia coli for production of the anti-malarial drug precursor amorpha-4,11-diene, Metabolic engineering 11, 13-19.
20. Tyo, K. E., Ajikumar, P. K., and Stephanopoulos, G. (2009) Stabilized gene duplication enables long-term selection-free heterologous pathway expression, Nature biotechnology 27, 760-765.
21. Gueldener, U., Heinisch, J., Koehler, G. J., Voss, D., and Hegemann, J. H. (2002) A second set of loxP marker cassettes for Cre-mediated multiple gene knockouts in budding yeast, Nucleic acids research 30, e23.
22. Lee, W. H., Seo, S. O., Bae, Y. H., Nan, H., Jin, Y. S., and Seo, J. H. (2012) Isobutanol production in engineered Saccharomyces cerevisiae by overexpression of 2-ketoisovalerate decarboxylase and valine biosynthetic enzymes, Bioproc Biosyst Eng 35, 1467-1475.
23. Kondo, T., Tezuka, H., Ishii, J., Matsuda, F., Ogino, C., and Kondo, A. (2012) Genetic engineering to enhance the Ehrlich pathway and alter carbon flux for increased isobutanol production from glucose by Saccharomyces cerevisiae, Journal of biotechnology 159, 32-37.
24. Chen, X., Nielsen, K. F., Borodina, I., Kielland-Brandt, M. C., and Karhumaa, K. (2011) Increased isobutanol production in Saccharomyces cerevisiae by overexpression of genes in valine metabolism, Biotechnology for biofuels 4, 21.
25. Brat, D., Weber, C., Lorenzen, W., Bode, H. B., and Boles, E. (2012) Cytosolic re-localization and optimization of valine synthesis and catabolism enables inseased isobutanol production with the yeast Saccharomyces cerevisiae, Biotechnology for biofuels 5, 65.
26. Maarse, A. C., Van Loon, A. P., Riezman, H., Gregor, I., Schatz, G., and Grivell, L. A. (1984) Subunit IV of yeast cytochrome c oxidase: cloning and nucleotide sequencing of the gene and partial amino acid sequencing of the mature protein, Embo J 3, 2831-2837.
27. Atsumi, S., Hanai, T., and Liao, J. C. (2008) Non-fermentative pathways for synthesis of branched-chain higher alcohols as biofuels, Nature 451, 86-89.
28. Connor, M. R., Cann, A. F., and Liao, J. C. (2010) 3-Methyl-1-butanol production in Escherichia coli: random mutagenesis and two-phase fermentation, Applied microbiology and biotechnology 86, 1155-1164.
29. Huo, Y. X., Cho, K. M., Rivera, J. G., Monte, E., Shen, C. R., Yan, Y., and Liao, J. C. (2011) Conversion of proteins into biofuels by engineering nitrogen flux, Nature biotechnology 29, 346-351.
30. Pfaffl, M. W. (2001) A new mathematical model for relative quantification in real-time RT-PCR, Nucleic acids research 29, e45.
31. Berges, T., Guyonnet, D., and Karst, F. (1997) The Saccharomyces cerevisiae mevalonate diphosphate decarboxylase is essential for viability, and a single Leu-to-Pro mutation in a conserved sequence leads to thermosensitivity, Journal of bacteriology 179, 4664-4670.
32. West, R. W., Jr., Chen, S. M., Putz, H., Butler, G., and Banerjee, M. (1987) GAL1-GAL10 divergent promoter region of Saccharomyces cerevisiae contains negative control elements in addition to functionally separate and possibly overlapping upstream activating sequences, Genes & development 1, 1118-1131.
33. Cartwright, C. P., Li, Y., Zhu, Y. S., Kang, Y. S., and Tipper, D. J. (1994) Use of Beta-Lactamase as a Secreted Reporter of Promoter Function in Yeast, Yeast 10, 497-508.
34. Paradise, E. M., Kirby, J., Chan, R., and Keasling, J. D. (2008) Redirection of flux through the FPP branch-point in Saccharomyces cerevisiae by down-regulating squalene synthase, Biotechnology and bioengineering 100, 371-378.
35. Paddon, C. J., Westfall, P. J., Pitera, D. J., Benjamin, K., Fisher, K., McPhee, D., Leavell, M. D., Tai, A., Main, A., Eng, D., Polichuk, D. R., Teoh, K. H., Reed, D. W., Treynor, T., Lenihan, J., Fleck, M., Bajad, S., Dang, G., Dengrove, D., Diola, D., Dorin, G., Ellens, K. W., Fickes, S., Galazzo, J., Gaucher, S. P., Geistlinger, T., Henry, R., Hepp, M., Horning, T., Iqbal, T., Jiang, H., Kizer, L., Lieu, B., Melis, D., Moss, N., Regentin, R., Secrest, S., Tsuruta, H., Vazquez, R., Westblade, L. F., Xu, L., Yu, M., Zhang, Y., Zhao, L., Lievense, J., Covello, P. S., Keasling, J. D., Reiling, K. K., Renninger, N. S., and Newman, J. D. (2013) High-level semi-synthetic production of the potent antimalarial artemisinin, Nature 496, 528-532.
36. Knight, S. A. B., Labbe, S., Kwon, L. F., Kosman, D. J., and Thiele, D. J. (1996) A widespread transposable element masks expression of a yeast copper transport gene, Genes & development 10, 1917-1929.
37. Westfall, P. J., Pitera, D. J., Lenihan, J. R., Eng, D., Woolard, F. X., Regentin, R., Horning, T., Tsuruta, H., Melis, D. J., Owens, A., Fickes, S., Diola, D., Benjamin, K. R., Keasling, J. D., Leavell, M. D., McPhee, D. J., Renninger, N. S., Newman, J. D., and Paddon, C. J. (2012) Production of amorphadiene in yeast, and its conversion to dihydroartemisinic acid, precursor to the antimalarial agent artemisinin, Proceedings of the National Academy of Sciences of the United States of America 109, E111-118.
38. Shao, Z., Zhao, H., and Zhao, H. (2009) DNA assembler, an in vivo genetic method for rapid construction of biochemical pathways, Nucleic acids research 37, e16.
39. Alper, H., Fischer, C., Nevoigt, E., and Stephanopoulos, G. (2005) Tuning genetic control through promoter engineering, Proceedings of the National Academy of Sciences of the United States of America 102, 12678-12683.
40. Nevoigt, E., Kohnke, J., Fischer, C. R., Alper, H., Stahl, U., and Stephanopoulos, G. (2006) Engineering of promoter replacement cassettes for fine-tuning of gene expression in Saccharomyces cerevisiae, Applied and environmental microbiology 72, 5266-5273.

1. An isolated linear expression cassette comprising:
a bidirectional promoter;
a first gene and a second gene that the genes are respectively and operably linked to one end of the bidirectional promoter;
a terminator located immediately next to end of each gene;
a 5′-Ty element and a 3′-Ty element respectively flanking 5′ end and 3′ end of the cassette; and
a dominant selection marker residing within the construct and being arranged in between the flanking 5′-Ty element and the 3′-element, wherein the cassette is capable of being integrated into genome of a transformed yeast through the Ty-elements upon being transported into the transformed cell.

2. The expression cassette of claim 1, wherein the dominant selection marker is an antibiotic selection marker, ble, kanMX, natMX, or hphMX.

3. The expression cassette of claim 1, wherein the dominant selection marker is coupled to and sandwiched in between a pair of site-specific recombination sequences.

4. The expression cassette of claim 3, wherein the pair of site-specific recombination sequences are loxP sites recognizable and capable of reacting with Cre recombinase or other equivalents to carry out recombinase-mediated marker recovery.

5. The expression cassette of claim 1, wherein the bidirectional promoter is formed by any two of the promoters derived from any functional promoter of native yeast cells, other organisms or synthetic promoters.

6. The expression cassette of claim 1, wherein the first gene and second gene are different.

7. The expression cassette of claim 1, wherein the first gene and second gene are ID11, tHMG1, ERG 8, ERG10, ERG12, ERG13, ERG19, ERG 20 ILV2, ILV5, ILV3, ARO10 and ADH7.

8. The expression cassette of claim 1, further comprising a sequence encoding mitochondrial targeting signal peptide.

9. A method of transforming yeasts by way of genome integration comprising:
transporting a plurality of linear expression cassettes into yeasts to produce transformed yeasts with at least one copy of the cassette integrated into genome of the yeasts, wherein each of the plurality of linear expression cassettes comprises:
a bidirectional promoter;
a first gene and a second gene that are each operably linked to a corresponding end of the bidirectional promoter;
a terminator located immediately next to the end of each gene;
a 5′-Ty element and a 3′-Ty element respectively flanking 5′ end and 3′ end of the cassette to enable integration of the cassette into genome of the transformed yeast upon transporting the cassette into the yeasts; and
a dominant selection marker residing within the construct and being arranged in between the flanking 5′-Ty element and the 3′-element;
growing the yeast with a medium containing a reagent capable of reacting with a marker expressed from the dominant selection marker by the transformed yeasts to generate a signal thereof; and
identifying the transformed yeasts based upon the generated signal.

10. The method of claim 9, wherein the marker is against an antibiotic being toxic to the yeasts and the reagent is the antibiotic that the identifying step comprises selecting the transformed yeast with a minimal number of expression cassette integrated based upon resistance capacity of the transformed yeasts against the antibiotic of a predetermined concentration by growing the transformed yeast in the presence of the antibiotic of the predetermined concentration, wherein the resistance capacity of the transformed yeast is substantially in positive correlation with the number of expression cassette integrated into the genome of the transformed yeasts.

11. The method of claim 9, wherein the first gene and the second gene of one linear expression cassette are different from another linear expression cassette.

12. The method of claim 9, wherein dominant selection marker is an antibiotic selection marker, ble, kanMX, natMX or hphMX.

13. The method of claim 9, wherein the dominant selection marker is coupled to and sandwiched in between a pair of site-specific recombination sequences.

14. The method of claim 13, wherein the pair of site-specific recombination sequences are loxP sites recognizable and capable of reacting with Cre recombinase or other equivalents to carry out recombinase-mediated marker recovery.

15. The method of claim 9, wherein the first gene and second gene are different and selected from the group consisting of ID11, tHMG1, ERG 8, ERG10, ERG12, ERG13, ERG19, ERG 20 ILV2, ILV5, ILV3, ARO10 and ADH7.

16. A method of transforming yeast by way of genome integration comprising:
transporting a plurality of linear expression cassettes into yeasts to produce transformed yeasts with at least one copy of the cassette integrated into the genome, each of the plurality of linear expression cassettes having a dominant selection marker against an antibiotic; and
selecting the transformed yeast with a minimal number of expression cassette integrated based upon resistance capacity of the transformed yeasts against the antibiotic of a predetermined concentration by growing the transformed yeast in the presence of the antibiotic of the predetermined concentration, wherein the resistance capacity of the transformed yeast is substantially in positive correlation with the number of expression cassette integrated into the genome of the transformed yeasts.

17. The method of claim 16, wherein the antibiotic against by the selection marker is phleomycin and the predetermined concentration is at least 40 μg/mL.

18. The method of claim 16, wherein the selection marker is ble, kanMX, natMX, hphMX or any modified selection marker derived thereof.
